(19) United States
(12) Patent Application Publication
(10) Pub. No.: US 2008/0161340 A1
(43) Pub. Date: Jul. 3, 2008

(54) Tetrahydroquinilinones, tetrahydronaphthyridones and derivatives thereof

(75) Inventors: Gary R. Gustafson, Ridgefield, CT (US); R. Paul Beckett, Yorktown Heights, NY (US)

Correspondence Address: CARA THERAPEUTICS, INC.;Intellectual Property Department, One Parrott Drive, Shelton, CT, 06484-4733, US

(73) Assignee: CARA THERAPEUTICS, INC., SHELTON, CT (US)

(21) Appl. No.: 12/004,134

(22) Filed: Dec. 19, 2007

Related U.S. Application Data
(60) Provisional application No. 60/876,081, filed on Dec. 20, 2006.
(60) Provisional application No. 60/904,645, filed on Mar. 2, 2007.

(51) Int. Cl. A61K 31/444; C07D 215/00; A61K 31/47; C07D 401/00; C07D 471/02; A61K 31/497

(52) U.S. Cl. 514/300; 546/156; 514/312; 514/314; 546/122; 546/167

(57) ABSTRACT

Tetrahydroquinolinone and tetrahydronaphthyridone cannabinoid receptor ligand compounds and stereoisomers, mixtures of stereoisomers, prodrugs, pharmaceutically acceptable salts, hydrates, solvates, acid salt hydrates, and isomorphic crystalline forms thereof are described. The compounds conform to the structure of formula I:

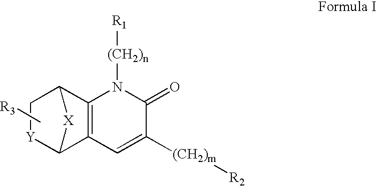

### RELATED APPLICATIONS

This application claims the benefit of the U.S. provisional application Ser. Nos. 60/876,081 filed Dec. 20, 2006 and 60/904,645 filed Mar. 2, 2007, the specifications of which are herein incorporated by reference in their entireties.

### BACKGROUND

Classical cannabinoids such as the marijuana derived cannabinoid Δ9-tetra-hydrocannabinol, (Δ9-THC) produce their pharmacological effects through interaction with specific cannabinoid receptors in the body. So far, two cannabinoid receptors have been characterized: CB1, a receptor found in the mammalian brain and peripheral tissues and CB2, a receptor found only in the peripheral tissues. Compounds that are modulators (including agonists, inverse agonists and antagonists) for one or both of these receptors have been shown to provide a variety of pharmacological effects. See, for example, Mackie, K., Cannabinoid receptors as therapeutic targets, Ann. Rev. Pharmacol. Toxicol. (2006) 46: 101-122; Pertwee, R. G., The therapeutic potential of drugs that target cannabinoid receptors or modulate the tissue levels or actions of endocannabinoids, AAP J. (2005) 7:E625-654; Pertwee, R. G., Pharmacology of cannabinoid CB1 and CB2 receptors, Pharmacology and Therapeutics (1997) 74:129-180; Di Marzo, V., Melck, D., Bisogno, T., DePetrocellis, L., Endocannabinoids: endogenous cannabinoid receptor ligands with neuromodulator action, Trends in Neuroscience (1998) 21:521-528.

### SUMMARY OF THE INVENTION

The present invention provides compounds having the structure of formula I,

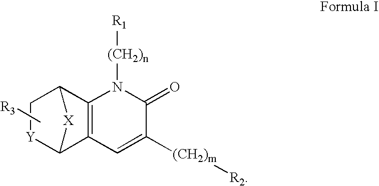

Also contemplated within the scope of the present invention are stereoisomers, mixtures of stereoisomers, prodrugs, pharmaceutically acceptable salts, hydrates, solvates, acid salt hydrates, and isomorphic crystalline forms of the compounds having the structure of formula I.

In formula I, the R₁ moiety can be hydrogen, an optionally substituted one to six-membered alkyl chain, an optionally substituted three to six-membered cycloalkyl, an optionally substituted three to six-membered cycloalkyloxy, SO₂R₄, COR₄, wherein R₄ is an optionally substituted one to four-membered alkyl chain, an optionally substituted alkoxy radical or a saturated or unsaturated optionally substituted 5-, 6- or 7-membered heterocycle optionally fused with a four- to eight-membered carbocycle. Alternatively, R₁ can be optionally substituted phenyl or naphthyl.

The R₂ moiety can be optionally substituted phenyl, optionally substituted three- to seven-membered cycloalkyl, optionally substituted three- to seven-membered cycloalkenyl, optionally substituted five- or six-membered heterocyclyl optionally fused with a four- to eight-membered carbocycle, optionally substituted alkoxy. Alternatively, R₂ can be

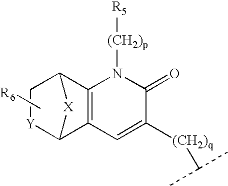

wherein R₅ is independently selected from R₁, R₆ is independently chosen from R₃, and the operators n, m, p and q are each independently 0, or an integer chosen from 1, 2, or 3.

The R₃ moiety can be hydrogen, amino, optionally substituted one to six-carbon atom straight or branched chain alkyl, optionally substituted one to six-carbon atom straight or branched chain alkoxy, optionally substituted one to six-carbon atom straight or branched chain alkylthio, three to six-membered cycloalkyl, optionally substituted phenyl, optionally substituted naphthyl, optionally substituted heterocyclyl, —SO₂R₇, —CONHR₇, —COR₇ or —COOR₇, wherein R₇ is one to four membered alkyl, phenyl, naphthyl or 5- or 6-membered heterocyclyl. In formula I, —X— is —CH₂—, —O—, —S—, —SO—, —SO₂—, or is absent; and —Y— is a carbon or a nitrogen atom.

Each of the optional substituents of the substituted moieties listed above is independently chosen from phenyl, heterocyclyl, straight or branched alkyl chain of one to six carbon atoms, one to six carbon atom straight or branched chain alkoxy, one to six carbon atom straight or branched chain alkylthio, oxo, hydroxyl, halo, amino, nitro, cyano, carboxyl, and amidino, except that oxo is not permitted as a substituent of phenyl or naphthyl.

### DETAILED DESCRIPTION OF THE INVENTION

The following definitions give the meaning of the listed terms a used herein:

Alkyl—a saturated branched or straight chain monovalent hydrocarbon radical, typically of up to about 6 carbon atoms. Thus, the term alkyl includes, but is not limited to, methyl, ethyl, n-propyl, isopropyl, n-butyl, t-butyl, n-pentyl, n-hexyl. A chain of 1-6 carbon atoms is also herein interchangeably designated as C₁-C₆ alkyl; a chain of 3 to 6 carbon atoms may be designated as C₃-C₆ alkyl and so forth.

Alkoxy—refers to an —O-alkyl substituent group linked through the oxygen atom and where the alkyl group is as defined above, such as for instance and without limitation, —O-methyl, O-ethyl, —O-propyl and so forth.

Cycloalkyl—a saturated or partially unsaturated monocyclic, polycyclic or bridged hydrocarbon ring system radical or linking group. A ring of 3 to 8 carbon atoms may be interchangeably designated as C₃-C₈ cycloalkyl; a ring of 3 to 8 carbon atoms may be designated by C₃-C₈ cycloalkyl and so forth. Cycloalkyl typically includes, but is not limited to, cyclopropyl, cyclobutyl, cyclopentyl, cyclohexyl, cyclohexenyl, cycloheptyl, cyclooctyl, 1,3,3-trimethylbicyclo[2.2.1]heptyl. In a substituted cycloalkyl ring, the substituent is bonded to ring carbon atom replacing a hydrogen atom.

Heterocyclyl—a saturated, partially unsaturated or unsaturated monocyclic, polycyclic or bridged hydrocarbon ring system radical or linking group, wherein one or more ring carbon atoms have been replaced with a heteroatom, each independently selected from N, O, or S. A heterocyclyl ring system further includes a ring system having up to 4 nitrogen atom ring members or a ring system having from 0 to 3 nitrogen atom ring members and 1 oxygen or sulfur atom ring member. The heterocyclic ring system can include more than one ring heteroatom, wherein one heteroatom is nitrogen and the other is selected from N, O, or S. A heterocyclyl radical is derived by the removal of one hydrogen atom from a single carbon or nitrogen ring atom. In addition, the sulfur atom can be in the +2 (—S—), +4 (—SO—) or +6 (—SO₂—) oxidation state.

Heterocyclyl rings can include multiple fused rings wherein one or more of the multiple rings can be aromatic or include one or more unsaturated bonds. Typical five-membered heterocyclyl radicals include, without limitation, furanyl, thiophenyl, pyrrolidinyl, pyrazolyl, thiazolinyl, thiazolyl, oxazolyl, and saturated derivatives such as tetrahydrofuranyl, tetrahydrothiophenyl, tetrahydrothiazolyl, and tetrahydrooxazolyl. Typical six-membered heterocyclyl radicals include, for instance, piperidinyl, piperazinyl, morpholinyl, thiomorpholinyl, dithianyl and trithianyl. Other useful heterocyclyl radicals include, for instance and without limitation, seven-membered heterocyclyl radicals, such as azepanyl, thiopanyl, diazepinyl, and triazopinyl; and eight-membered heterocyclyl radicals, such as azecanyl, thiocanyl, oxocanyl, diazecanyl, and triazocanyl. Multicyclic heterocyclyl radicals include, without limitation, 5-, 6- or 7-membered heterocyclic rings fused with a four to eight-membered carbocycle. Each heterocyclic and carbocyclic ring can include one or more unsaturated bonds.

Heterocyclyl—includes, but is not limited to, furyl, thienyl, 2H-pyrrole, 2-pyrrolinyl, 3-pyrrolinyl, pyrrolidinyl, pyrrolyl, 1,3-dioxolanyl, oxazolyl, thiazolyl, imidazolyl, 2-imidazolinyl, imidazolidinyl, 2-pyrazolinyl, pyrazolidinyl, pyrazolyl, isoxazolyl, isothiazolyl, oxadiazolyl, triazolyl, thiadiazolyl, tetrazolyl, 2H-pyranyl, 4H-pyranyl, pyridinyl, piperidinyl, 1,4-dioxanyl, morpholinyl, 1,4-dithianyl, thiomorpholinyl, pyridazinyl, pyrimidinyl, pyrazinyl, piperazinyl, azepanyl, diazepinyl, indolizinyl, indolyl, isoindolyl, 3H-indolyl, indolinyl, benzo[b]furyl, benzo[b]thienyl, 1H-indazolyl, benzimidazolyl, benzothiazolyl, purinyl, 4H-quinolizinyl, quinolinyl, isoquinolinyl, cinnolinyl, phthalzinyl, quinazolinyl, quinoxalinyl, 1,8-napthyridinyl, pteridinyl, quinuclidinyl.

Alkylheterocyclyl—an optionally substituted heterocyclyl group carrying an obligatory C₁-C₄ alkyl substitution bonded to a carbon atom of the alkyl substituent.

Aminosulfonylalkyl—a radical of the formula —NHSO₂-alkyl. Sulfonylaminoalkyl—a linking group of the formula —SO₂NH-alkyl- or a radical of the formula —SO₂N(alkyl)₂. Alkylcarbamoyl—a linking group of the formula -alkyl-C(O)NH—or a radical of the formula -alkyl-C(O)NH₂. Carbamoylalkyl—a linking group of the formula —NHC(O)-alkyl- or a radical of the formula —NHC(O)-alkyl.

Halo—fluoro, chloro, bromo or iodo. Carboxy—a radical of the formula —COOH. Hydroxyl—a radical of the formula —OH. Cyano—a radical of the formula —C—N. Oxo—a linking group of the formula —CO— in which the oxygen atom is double bonded to the carbon atom.

Amino—a radical of the formula —NH₂ or a linking group of the formula —NH—. Aminoalkyl—a radical of the formula —NH-alkyl or —N(alkyl)₂.

The present invention provides compounds and stereoisomers, mixtures of stereoisomers, prodrugs, pharmaceutically acceptable salts, hydrates, solvates, acid salt hydrates, and isomorphic crystalline forms of the compounds of formula I:

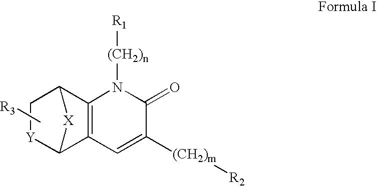

As used herein, the terms: a single compound, salt, polymorph, isomer, solvate are also interchangeably referred to in the plural form (i.e. compounds, salts, polymorphs, isomers and solvates).

In particular embodiments of compounds of the present invention, R₁ is hydrogen, an optionally substituted one to six-membered alkyl chain, an optionally substituted three to six-membered cycloalkyl, an optionally substituted three to six-membered cycloalkyloxy, SO₂R₄, COR₄, wherein R₄ is an optionally substituted one to four-membered alkyl chain, a saturated or unsaturated optionally substituted 5-, 6- or 7-membered heterocycle, or an optionally substituted alkoxy radical. Alternatively, R₁ can be phenyl, or naphthyl.

In other embodiments the R₂ moiety can be optionally substituted phenyl, optionally substituted three- to seven-membered cycloalkyl, optionally substituted three- to seven-membered cycloalkenyl, optionally substituted five- or six-membered heterocyclyl optionally fused with a four- to eight-membered carbocycle, optionally substituted alkoxy. Alternatively, in still other embodiments R₂ can be

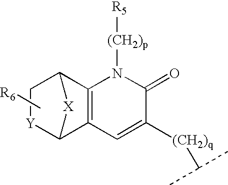

wherein R₅ is independently chosen from R₁ above, R₆ is independently chosen from R₃ above, n, m, p and q are each independently 0, 1, 2 or 3. In some embodiments R₁ is identical to R₁. In other embodiments R₆ is identical to R₃. In still other embodiments R₁ is identical to R₁ and R₆ is identical to R₃. In each of these embodiments, p can be identical to n and/or q can be identical to m. In one particular embodiment n, m, p and q are each zero. In another particular embodiment n and p are each 1, and m and q are each zero.

In certain embodiments of compounds of the present invention, the R₃ moiety is hydrogen, amino, optionally substituted one to six-carbon atom straight or branched chain alkyl, optionally substituted one to six-carbon atom straight or branched chain alkoxy, optionally substituted one to six-carbon atom straight or branched chain alkylthio, three to six-membered cycloalkyl, optionally substituted phenyl, optionally substituted naphthyl, optionally substituted heterocyclyl, SO₂R₇, CONHR₇, COR₇ or COOR₇; wherein R₇ is one to four-membered alkyl, phenyl, naphthyl or 5- or 6-membered heterocyclyl.

In particular embodiments of compounds of the present invention, the moiety —X— in formula I is —CH₂—, —O—, —S—, —SO—, —SO₂—, or is a bond; and the moiety Y is carbon or nitrogen atom.

Each of the above-listed optionally substituted moieties is substituted with one, two or three substituents. Each substituent is independently chosen from one to six-membered alkyl, one to six-membered cycloalkyl, one to six-membered alkoxy, alkylthio, phenyl, heterocyclyl, halo, —OH, —NH₂, oxo, —NO₂, —CN, —COOH, thiol, alkylthio, sulfonyl, sulfinyl or sulfanyl and amidino, except that oxo is not permitted as a substituent of phenyl or naphthyl.

The optionally substituted one to six-membered alkyl of R₁ can be any substituted or unsubstituted one to six-membered alkyl, such as, without limitation, methyl, ethyl, propyl, butyl, iso-butyl, tert-butyl, pentyl, 1-methylbutyl, 2-methylbutyl, 3-methylbutyl, 1-ethylpropyl or 2-ethylpropyl. The optionally substituted three to six-membered cycloalkyl of R₁ can be any substituted or unsubstituted cyclopropyl, cyclobutyl, cyclopentyl or cyclohexyl.

The optionally substituted heterocyclyl of R₁, such as for instance, piperidinyl, can be any substituted or unsubstituted 1-, 2-, -3, or 4-piperidinyl. As a further example, an optionally substituted piperazinyl of R₁ can be any substituted or unsubstituted 1-, 2- or 3-piperazinyl. When R₁ is 1-piperidinyl or 1-piperazinyl, n is 1, or 2. R₁ can also be SO₂R₄ or COR₄ wherein R₄ is an optionally substituted one to four-membered alkyl, optionally substituted one to four-membered alkoxy, or optionally substituted one to four-membered alkyloxy. The R₄ one to four-membered alkyl can be any substituted or unsubstituted one to six-membered alkyl, such as, without limitation, methyl, ethyl, propyl, butyl, iso-butyl, tert-butyl, pentyl, 1-methylbutyl, 2-methylbutyl, 3-methylbutyl, 1-ethylpropyl or 2-ethylpropyl, each of which can be optionally singly or multiply substituted with one or more independently selected fluoro-, chloro-, or bromo-substituents.

The R₄ moiety can also be any alkoxy moiety including, but not limited to methoxy, ethoxy, propyloxy, and butyloxy. The R₄ moiety can also be any hydroxyl-, oxo-, carboxy-, nitro-, amino- or thio-substituted methyl, ethyl, propyl, butyl, iso-butyl, tert-butyl, pentyl, 1-methylbutyl, 2-methylbutyl, 3-methylbutyl, 1-ethylpropyl or 2-ethylpropyl. In addition, R₄ can be any haloalkoxy moiety including, but not limited to single or independently selected multiple fluoro, chloro, or bromo-substituted methoxy, ethoxy, propyloxy, or butyloxy; or R₄ can be a hydroxyl, carboxy, nitro, amino or thiol.

The optionally substituted phenyl or naphthyl of R₂ can be any singly-substituted, multiply-substituted or unsubstituted phenyl or naphthyl including, but not limited to phenyl, 2-, 3-, or 4-substituted phenyl, or benzyl, 2-, 3-, or 4-substituted benzyl, wherein the substituent is a methyl, fluoro, chloro, bromo, hydroxyl, carboxy, nitro, amino or thiol, or any independently selected combination of the above substituents.

An optionally substituted five to seven-membered cycloalkyl of R₂ as used herein includes saturated and partially unsaturated five to seven-membered cycloalkyl. The substituted partially unsaturated five to seven-membered cycloalkyl of R₂ can be any substituted partially unsaturated five to seven-membered cycloalkyl such as cyclopentyl, cyclohexyl or cycloheptyl, substituted with, for example and without limitation, one or more independently selected methyl-, fluoro-, chloro-, bromo-, hydroxyl-, oxo-, carboxy-, nitro-, amino- or thiol.

The optionally substituted five- or six-membered heterocycles of R₁ and R₂ can be any substituted or unsubstituted, saturated or unsaturated five- or six-membered heterocycle having either (i) one, two or three nitrogens, or (ii) an oxygen or a sulfur alone or in combination with one, two or three nitrogens; these heterocycles include, but are not limited to furan, thiophene, pyrrole, pyrroline, pyrrolidine, oxazole, thiazole, imidazole, imidazoline, imidazolidine, pyrazole, pyrazoline, pyrazolidine, isoxazole, isothiazole, oxadiazole, triazole, thiadiazole, pyran, pyridine, piperidine, morpholine, thiomorpholino, pyridazine, pyrimidine, pyrazine, piperazine, and triazine.

The optionally substituted alkoxy of R₂ can be any substituted or unsubstituted alkoxy, such as but not limited to methoxy, ethoxy, propyloxy, or butyloxy, substituted with methyl-, fluoro-, chloro-, bromo-, hydroxyl-, oxo-, carboxy-, nitro-, amino- or thiol.

The moiety, R₂ can also be R_{d} wherein R_{d} is the structure:

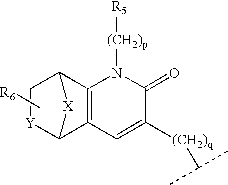

such that this particular compound of the invention is a dimeric structure comprising an R_{d} unit bonded to a structure of formula I at R₂ through the two optional alkyl chains: (CH₂)_{q} and (CH₂)ₘ. In one embodiment wherein the molecule is a homodimer, R₅ is identical to R₁, and R₆ is identical to R₃. In another embodiment p=n, q=m. In another embodiment —X— is absent. In a particular embodiment, the compound is a symmetrical homodimer, wherein R₅ is identical to R₁, and R₆ is identical to R₃ and p=n, q=m, and the optional bridging moiety, —X— is absent.

In certain embodiments the compounds of the invention are agonists for a mammalian G protein-coupled receptor (GPCR), particularly a human GPCR. In preferred embodiments the compounds of the invention are agonists for a mammalian cannabinoid CB2 receptor, particularly the human CB2 receptor. In particular preferred embodiments the compounds of the invention are full agonists for the human CB2 receptor. That is, when contacted with the human CB2 receptor at sufficiently high concentrations above the EC₅₀, these compounds are capable of fully inducing the activity of the receptor.

In some embodiments, the compounds of the invention are selective agonists for a mammalian CB2 receptor and do not function as agonists for the CB1 receptor of that mammal. In particular embodiments, the compounds are selective agonists for the human CB2 receptor while having little or no agonist activity for the human CB1 receptor. In particular embodiments, the compounds of the invention are salts, solvates, esters, stereoisomers or racemates of a compound of formula I or preferably a pharmaceutically acceptable salt, solvate, ester, stereoisomer or racemate of a compound of formula I.

The compounds of the present invention are believed to be ligands for a mammalian CB2 receptor. In certain embodiments, the compounds of the present invention have an EC₅₀ of less than about 500 nM for a mammalian CB2 receptor, particularly, the human CB2 receptor. In preferred embodiments, the compounds have an EC₅₀ of less than about 100 nM for the human CB2 receptor. In other more preferred embodiments, the compounds have an EC₅₀ of less than about 50 nM for the human CB2 receptor. In still more preferred embodiments, the compounds have an EC₅₀ of less than about 20 nM for the human CB2 receptor. In yet more preferred embodiments, the compounds have an EC₅₀ of less than about 5 nM for the human CB2 receptor. Optimally, the compounds have an EC₅₀ of less than about 1 nM for the human CB2 receptor.

### Synthetic Methods

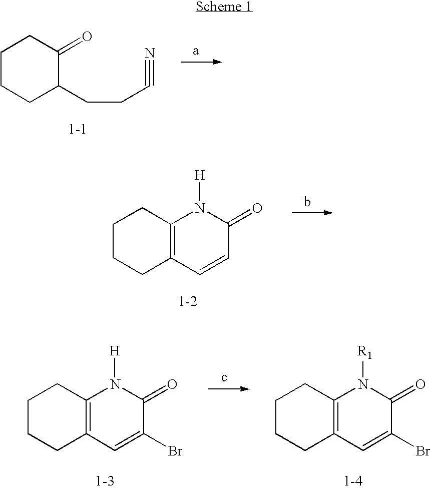

Reagents and conditions for reactions in Scheme 1 are as follows: a) H₂SO₄, 0° C.; b) NBS in DMF or PBr₃ in DCM; c) Alkyl halide, NaH or LiH in THF in a microwave oven at 160° C. for 10 min.

A method for preparing compounds of formula I where m and n are each 1 is shown in scheme 1. Cyclization of 3-(2-oxocyclohexyl)propanenitrile is accomplished through the use of an acid such as sulfuric acid. Bromination of 1-2 is performed by using reagents such as N-bromosuccinimide in an aprotic solvent, for instance DMF. Alkylation of the nitrogen of 1-3 occurs through the use of a base, for instance LiH, and a primary halide in an aprotic solvent such as DMF at elevated temperatures, for example, 150° C. in a microwave.

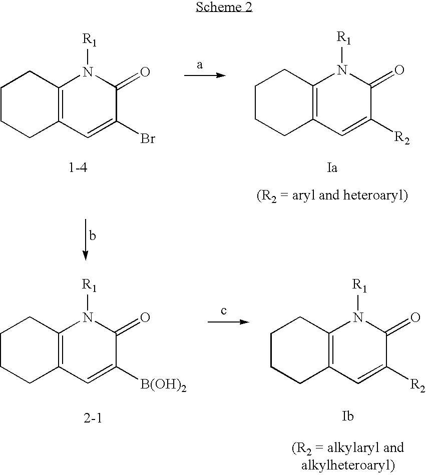

Reagents and conditions for reactions in Scheme 2 are as follows: a) Arylboronic acid, Pd(PPh₃)₄, Na₂CO₃; b) n-BuLi, tributyl borate; c) alkyl halide, Pd(PPh₃)₄, Na₂CO₃.

Intermediate 1-4 can be used to synthesize final compounds of the formula Ia or Ib as shown in scheme 2. Intermediate 1-4 can be placed under Suzuki conditions to attach an aryl or heteroaryl moiety through use of the appropriate catalyst, such as palladium-tetrakis(triphenylphosphine), an aqueous base, and an aryl or heteroaryl boronic acid in an aprotic solvent, such as THF or toluene, at elevated temperatures, for instance 160° C. in a microwave. This yields compounds of the formula Ia. Alternatively, intermediate 1-4 can be converted to a boronic acid through the use of n-BuLi and a borate ether, such as tributyl borate, in an aprotic solvent, for example THF, at low temperatures, such as −78° C. After quenching with acid, the boronic acid 2-1 is produced. Intermediate 2-1 can be placed under Suzuki conditions to attach an alkylaryl or alkylheteroaryl moiety through use of the appropriate catalyst, such as palladium-tetrakis(triphenylphosphine), an aqueous base, and a halide, for instance benzyl bromide in an aprotic solvent, such as THF or toluene, at elevated temperatures, for instance 160° C. in a microwave. This yields compounds of the formula Ib.

Compounds of formula Ic and Id can be prepared by the methods described in the general synthetic Schemes 3-6 described below.

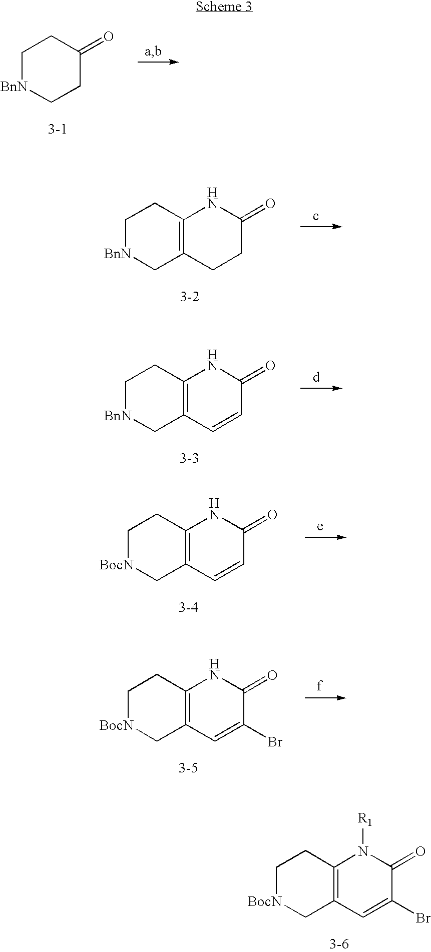

Reagents and conditions for reactions in Scheme 3 are as follows: a) Pyrrolidine, microwave 160° C., 10 min; b) acrylamide, pTsOH, microwave 160° C., 10 min; c) Br₂, AcOH, microwave 150° C., 5 min.; d) Pd(OH)₂, H₂, (Boc)₂O; e) NBS in DMF or PyBr₃ in DCM; f) Alkyl halide, NaH or LiH in THF in a microwave oven at 160° C., 10 min.

Starting with the 1-benzylpiperidin-4-one, reaction with pyrrolidine at elevated temperatures, for example, 160° C. in a microwave is performed. After removal of volatiles, acylamide is added to the reaction along with a catalytic acid, such as p-toluene sulfonic acid, an aprotic solvent, for example dioxane, and the reaction is heated at elevated temperatures, such as 160° C. in a microwave. This yields intermediate 3-2 which can be oxidized using such reagents as bromine to provide 3-3. Exchange of the protecting group can be done by hydrogenolysis of the benzyl group, for example with Pd(OH)₂ and H₂, in the presence of (Boc) 20 to yield intermediate 3-4. Bromination of 3-4 is performed by using reagents such as pyridinium tribromide in an aprotic solvent, for instance DCM to provide the intermediate 3-5. Alkylation of the nitrogen of 3-5 occurs through the use of a base, for instance LiH, and a primary halide in an aprotic solvent such as DMF at elevated temperatures, for example, 150° C. in a microwave to yield 3-6.

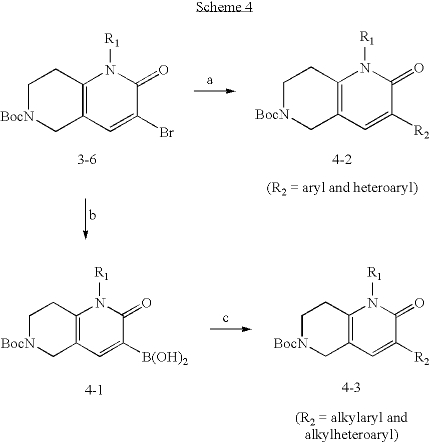

Reagents and conditions for reactions in Scheme 4 are as follows: a) Arylboronic acid, Pd(PPh₃)₄, Na₂CO₃; b) n-BuLi, tributyl borate; c) alkyl halide, Pd(PPh₃)₄, Na₂CO₃.

Intermediate 3-6 can be reacted to produce intermediates 4-2 and 4-3 using the same method as was detailed in the description of Scheme 2 to produce Ia and Ib from intermediate 1-4.

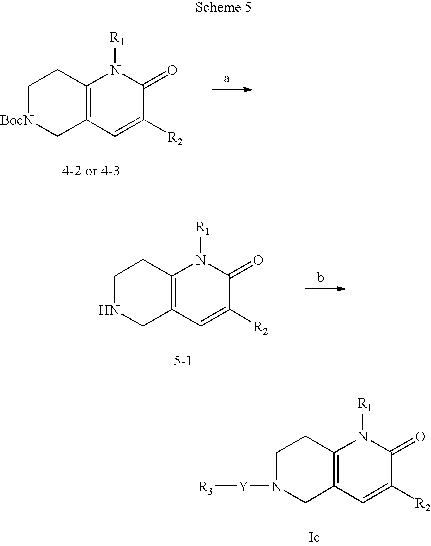

Reagents and conditions for reactions in Scheme 5 are as follows: a) TFA in DCM; b) Sulfonyl halide or acid halide, DIPEA in THF.

The intermediates 4-2 and 4-3 can be deprotected under acidic conditions, such as TFA in DCM to produce intermediate 5-1. This compound can be treated with an acid halide, acid anhydride or sulfonyl halide in dichloromethane in the presence of base (such as aqueous inorganic bicarbonate, or a tertiary amine base and/or DMAP) or under reducing conditions with an aldehyde (such as NaBH(OAc)₃).

### EXAMPLES

All reactions involving moisture sensitive compounds were carried out under an anhydrous nitrogen or argon atmosphere. All reagents were purchased from commercial sources and used without further purification. Unless otherwise noted, the starting materials used in the examples were obtained from readily available commercial sources or synthesized by standard methods known to those skilled in the art of organic synthesis. Normal phase chromatography and reverse phase chromatography was performed on an ISCO CombiFlash Companion.

Compounds were characterized by their HPLC-Electrospray/chemical ionization mass spectra (HPLC ESCI-MS) on a Waters HPLC-MS system (Waters Corp., Milford, Mass.) equipped with a 2767 Sample Manager, 2545 Binary Gradient Module, SFO System Fluidics Organizer, 2996 Photodiode Array Detector and 3100 Mass Detector. Data was collected across a range of wavelengths from 220 nm to 280 nm and in positive ESCI mode. Spectra were scanned from 100-1400 atomic mass units. The HPLC column was a Waters XBridge C18 3.5 um 4.6×30 mm; eluents were A: water with 0.1% formic acid and B: acetonitrile with 0.1% formic acid. Gradient elution was from 5% B to 95% B over 2.3 minutes with an initial hold of 0.2 minutes and a final hold at 95% B of 0.5 minutes. Total run time was 4 minutes.

Normal phase chromatography was done on an ISCO CombiFlash Companion and reverse phase chromatography was done on a Waters AutoPurification System with 3100 Mass Detector. Mass spectra (MS) were determined on the Waters SQ Detector/3100 Mass Detector using electrospray techniques. Unless otherwise noted, the materials used in the examples were obtained from readily available commercial sources or synthesized by standard methods known to those skilled in the art of organic synthesis.

Abbreviations used herein:
Bn Benzyl
n-BuLi n-Butyl lithium
Boc tert-butyloxycarbonyl
(Boc)₂O Di-tert-butyl dicarbonate
Celite Diatomaceous earth
DCM Dichloromethane
DMF Dimethylformamide
DIPEA Diisopropylethylamine
DMAP 2,6-dimethylaminopyridine
EtOAc Ethyl acetate
EtOH Ethanol
g Grams
H₂ Hydrogen
H₂O Water
H₂SO₄ Sulfuric acid
Hex Hexane(s)
HCl Hydrochloric acid
LiH Lithium hydride
MeOH Methanol
mg Milligrams
mL Milliliter
mmol Millimole
uL Microliter
μmol Micromole
NaBH(OAc)₃ Sodium triacetoxyborohydride
NaH Sodium hydride
NaHCO₃ Sodium bicarbonate
Na₂SO₄ Sodium sulfate
NBS N-bromosuccinimide
Pd(OH)₂ Palladium hydroxide
pTsOHp-Toluenesulfonic acid
PyBr₃ Pyridinium tribromide
TFA Trifluoroacetic acid
THF Tetrahydrofuran

### Preparation of Intermediate A: 3-bromo-1-(cyclobutylmethyl)-5,6,7,8-tetrahydroquinolin-2(1H)-one

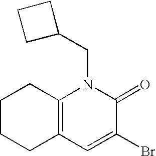

### Step 1: Preparation of 5,6,7,8-tetrahydroquinolin-2(1H)-one

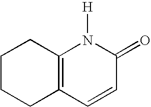

2-(2-cyanoethyl)cyclohexanone (1.5 g, 10 mmol) was added dropwise to 10 mL of ice cold H₂SO₄ in a 40 mL vial. Once the addition was complete, the vial was capped and shaken for 3 hours while warming to room temperature. The reaction was then poured over ice and extracted with 5 mL DCM. The aqueous layer was then neutralized with ammonia resulting in the formation of a white precipitate. The mixture was then extracted twice with 5 mL DCM. The combined organic layers were dried with Na₂SO₄, filtered through celite and evaporated. This yielded a white solid, 5,6,7,8-tetrahydroquinolin-2(1H)-one, (1.0 gm) that was used without further purification. MS [M+H]⁺=150.08.

### Step 2: Preparation of 3-bromo-5,6,7,8-tetrahydroquinolin-2(1H)-one

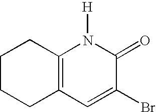

5,6,7,8-tetrahydroquinolin-2(1H)-one (200 mg, 1.3 mmol) was dissolved in 2 mL of DMF. N-bromosuccinimide (250 mg, 1.4 mmol) was added and the reaction was stirred at room temperature for 2 hours. Two milliliters of H₂O and 2 mL of EtOAc was added to the reaction. The top organic layer was removed and the aqueous layer was extracted with 1 mL of EtOAc. The combined organic layers were dried with Na₂SO₄, filtered through celite and evaporated. This yielded a light brown solid, 3-bromo-5,6,7,8-tetrahydroquinolin-2(1H)-one, (151 mg) that was used without further purification. MS [M+H]⁺=227.99.

### Step 3: Preparation of: 3-bromo-1-(cyclobutylmethyl)-5,6,7,8-tetrahydroquinolin-2(1H)-one

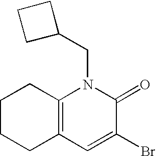

In a 2.0-5.0 mL microwave reaction vial with a stir bar was placed 3-bromo-5,6,7,8-tetrahydroquinolin-2(1H)-one (750 mg, 3.29 mmol) in 3 mL DMF and LiH (79 mg, 9.86 mmol). Hydrogen gas evolved from the reaction. After 5 minutes, (cyclobutyl)methyl bromide (735 mg, 4.93 mmol) was added by pipette and the vial was capped. The reaction was heated to 160° C. for 10 minutes on the Biotage microwave. The reaction was diluted with 5 mL of water and 5 mL of EtOAc. The mixture was vortexed and the aqueous layer removed. The aqueous layer was extracted again with 5 mL of EtOAc. The organic layers were combined, dried with Na₂SO₄, and filtered. Silica gel was added to the vial and the residue adsorbed onto silica by evaporating the solvent. The resulting silica was packed into an empty cartridge. The sample was purified by Flash chromatography (gradient elution, 5% EtOAc/Hex to 50% EtOAc/Hex). The appropriate fractions were combined to yield a light yellow oil, 3-bromo-1-(cyclobutylmethyl)-5,6,7,8-tetrahydroquinolin-2(1H)-one, (692 mg). MS [M+H]⁺=296.06

### Preparation of Intermediate B: 3-bromo-1-(cyclopropylmethyl)-5,6,7,8-tetrahydroquinolin-2(1H)-one

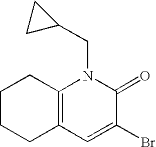

This compound was prepared using the procedure outlined in the synthesis of Intermediate A, with substitution of (cyclopropyl)methyl bromide for (cyclobutyl)methyl bromide in step 3. This yielded a light yellow oil, 3-bromo-1-(cyclopropylmethyl)-5,6,7,8-tetrahydroquinolin-2(1H)-one. MS [M+H]⁺=282.04.

### Preparation of Intermediate C: 3-bromo-1-(cyclohexylmethyl)-5,6,7,8-tetrahydroquinolin-2(1H)-one

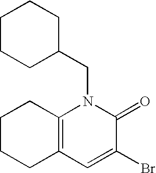

This compound was prepared using the procedure outlined in the synthesis of Intermediate A, with substitution of (cyclohexyl)methyl bromide for (cyclobutyl)methyl bromide in step 3. This yielded a light yellow oil, 3-bromo-1-(cyclohexylmethyl)-5,6,7,8-tetrahydroquinolin-2(1H)-one. MS [M+H]⁺=324.09.

### Preparation of Intermediate D: 1-(cyclobutylmethyl)-2-oxo-1,2,5,6,7,8-hexahydroquinolin-3-ylboronic acid

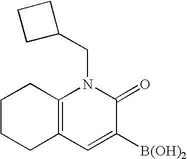

3-bromo-1-(cyclobutylmethyl)-5,6,7,8-tetrahydroquinolin-2(1H)-one (180 mg, 608 μmole) was dissolved in 1 ml of THF in a 2-dram vial containing a stir bar. The vial was cooled to −75° C. using a dry ice/acetone bath. To this mixture was added 2.5M n-BuLi (268 μL, 669 μmole). After the reaction was stirred for 1 hr. at −75° C., tributyl borate (396 μL, 669 μmole) was added and the reaction was allowed to warm to room temperature. The reaction was hydrolyzed with 1 mL of 10% HCl/water. The reaction mixture was extracted twice with 1 mL of EtOAc. The combined organic layers were dried with Na₂SO₄, filtered and evaporated. The resulting brown oil was used without purification. MS [M+H]⁺=262.15.

### Preparation of Intermediate E: 1-(cyclopropylmethyl)-2-oxo-1,2,5,6,7,8-hexahydroquinolin-3-ylboronic acid

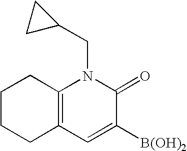

This compound was prepared using the procedure outlined in the synthesis of Intermediate D, with substitution of 3-bromo-1-(cyclopropylmethyl)-5,6,7,8-tetrahydroquinolin-2(1H)-one for 3-bromo-1-(cyclobutylmethyl)-5,6,7,8-tetrahydroquinolin-2(1H)-one. This yielded a brown oil that was used without further purification. MS [M+H]⁺=248.14

### Preparation of Intermediate F: tert-butyl 3-bromo-1-(cyclopropylmethyl)-2-oxo-1,2,7,8-tetrahydro-1,6-naphthyridine-6(5H)-carboxylate

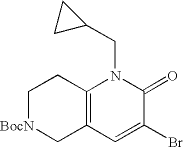

### Step 1: Preparation of 6-benzyl-3,4,5,6,7,8-hexahydro-1,6-naphthyridin-2(1H)-one

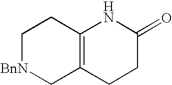

N-Boc pyridone (3.98 g, 20 mmol) and pyrrolidine (6.68 mL, 80 mmol) were added to 20 mL microwave vial along with a stirbar. The vial was capped and the reaction was heated neat for 10 minutes at 160° C. in the microwave. The reaction became brown. The vial was uncapped and 3 mL of toluene was added to the vial. All the volatiles were removed on the rotovap. The residue was used without further purification. To the residue (in a 20 mL microwave vial) was added pTsOH (76 mg, 0.4 mmol) and acrylamide (2.84 g, 40 mmol). About 5 mL of dioxane was added to get the volume high enough for absorption of microwaves. The vial was capped and the reaction heated for 15 minutes at 150° C. The reaction was partitioned between 10 mL EtOAc and 10 mL water. The layers were separated and the water extracted again with 5 mL of EtOAc and the organic layer were combined, dried with Na₂SO₄, filtered through a glass plug and evaporated. LCMS showed the reaction was complete so a second iteration in the microwave was unnecessary. This yielded a light orange solid, 6-benzyl-3,4,5,6,7,8-hexahydro-1,6-naphthyridin-2(1H)-one, (4.5 g) that was used without further purification. MS [M+H]⁺=243.14.

### Step 2: Preparation of 6-benzyl-5,6,7,8-tetrahydro-1,6-naphthyridin-2(1H)-one

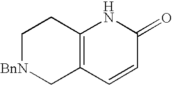

The Step 1 product (3.0 gr, 12.38 mmol) was placed in a 2-5 mL microwave vial and dissolved in 3 mL of glacial acetic acid. The brown solution was stirred with a stir bar. Bromine (634 μL, 12.38 mmol) was dissolved in 1 mL of glacial acetic acid creating a maroon solution. The bromine solution was dripped slowly over 5 minutes into the solution of the step one product. A precipitate would form as the bromine solution hit the product solution. It would quickly dissolve with stirring. Once all of the bromine was added, the microwave vial was capped. The reaction was placed in the microwave for 5 minutes at 150° C. When complete, the solution was poured into a 250 mL Erlenmeyer flask. Some solid was left behind. Saturated bicarbonate solution was slowly added to this solid (vigorous effervescence occurred). Once all the solid was dissolved, the solution was added to the rest of the solution in the Erlenmeyer. Solid bicarbonate was added in small aliquots until no more bubbling occurred upon addition. The pH of the solution was tested to make sure it was basic. There was much brown solid precipitated. The aqueous solution was extracted 2×10 mL with DCM. The combined organic layers were dried with Na₂SO₄, filtered through celite and evaporated. The residue was purified by Flash chromatography using EtOAc as eluent. This yielded a white/yellow solid, 6-benzyl-5,6,7,8-tetrahydro-1,6-naphthyridin-2(1H)-one, (2.42gr). MS [M+H]⁺=241.13.

### Step 3: Preparation of tert-butyl 2-oxo-1,2,7,8-tetrahydro-1,6-naphthyridine-6(5H)-carboxylate

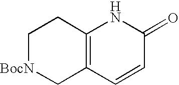

The Step 2 product (2 g, 8.32 mmol) was weighed into a Parr shaker bottle and dissolved in 20 mL of EtOH. Hunigs base (4.34 mL, 25 mmol), (Boc) 20 (2.36 g, 10.8 mmol) and catalyst (0.234 g, 1.7 mmol) were added. The bottle was placed on the Parr shaker and flushed 3× with H₂. The reaction was placed under 90 psi H₂ and left over the weekend. The reaction was filter through a glass frit packed with celite. The bottle was rinsed 3× with 5 mL MeOH and passed through the frit as well. The volatiles were evaporated and the residue dissolved in 10 mL of DCM. The solution was extracted with sat. bicarbonate. The organic layer was dried with sodium sulfate, filtered through a glass wool plug and the volatiles evaporated. This yielded a white solid, tert-butyl 3-bromo-2-oxo-1,2,7,8-tetrahydro-1,6-naphthyridine-6(5H)-carboxylate, (2.05 g). MS [M+H]⁺=251.13.

### Step 4: Preparation of tert-butyl 3-bromo-2-oxo-1,2,7,8-tetrahydro-1,6-naphthyridine-6(5H)-carboxylate

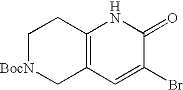

The Step 3 product (100 mg, 0.4 mmol) dissolved in 2 mL of DCM in a 20 mL scintillation vial containing a stir bar. Big chunks of the pyridinium tribromide (142 mg, 0.4 mmol) have to be crushed up to make them dissolve. The reaction was checked by LCMS after everything dissolved (5 minutes) and the starting material was gone. 5 mL of saturated bicarbonate was added to the orange solution. There was a great deal of bubbling and the organic layer became yellow. The organic layer was removed to a new 20 mL vial and dried with sodium sulfate, filtered through a glass wool plug and evaporated. The yellow/brown oil was used without further purification. MS [M+H]⁺=329.04.

### Step 5: Preparation of tert-butyl 3-bromo-1-(cyclopropylmethyl)-2-oxo-1,2,7,8-tetrahydro-1,6-naphthyridine-6(5H)-carboxylate

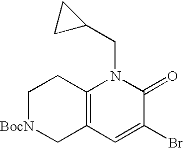

The product from Step 4 (assume 0.4 mmol) in a 0.5-2 mL microwave reaction vial with a stir bar was placed dissolved in 1 mL DMF and LiH (8 mg, 1 mmol). The reaction bubbled. After 5 minutes, (cyclopropyl)methyl bromide (58 μL, 0.6 mmol) was added and the vial was capped. The reaction was heated to 160° C. for 10 minutes on the Biotage microwave. The reaction was diluted with 2 mL of water and 2 mL of EtOAc. The mixture was vortexed and the aqueous layer removed. The aqueous layer was extracted again with 2 mL of EtOAc. The organic layers were combined, dried with Na₂SO₄, and filtered. Silica gel was added to the vial and the residue adsorbed onto silica by evaporating the solvent. The resulting silica was packed into an empty cartridge.

Purification of the sample was on the Biotage using a 12 gr column. A gradient that of from 10% EtOAc/Hex to 80% EtOAc/Hex was used. Fractions were combined and evaporated. This yielded a light yellow oil, tert-butyl 3-bromo-1-(cyclopropylmethyl)-2-oxo-1,2,7,8-tetrahydro-1,6-naphthyridine-6(5H)-carboxylate, (39 mg). MS [M+H]⁺=383.09.

### Preparation of Intermediate G: tert-butyl 3-bromo-1-(cyclobutylmethyl)-2-oxo-1,2,7,8-tetrahydro-1,6-naphthyridine-6(5H)-carboxylate

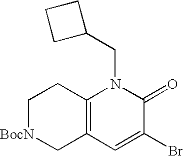

This compound was prepared using the procedure outlined in the synthesis of Intermediate F, with substitution of (cyclobutyl)methyl bromide for (cyclopropyl)methyl bromide in step 5. This yielded a yellow oil, tert-butyl 3-bromo-1-(cyclobutylmethyl)-2-oxo-1,2,7,8-tetrahydro-1,6-naphthyridine-6(5H)-carboxylate. MS [M+H]⁺=397.10.

### Example 1

### Preparation of 1-(cyclobutylmethyl)-3-phenyl-5,6,7,8-tetrahydroquinolin-2(1H)-one

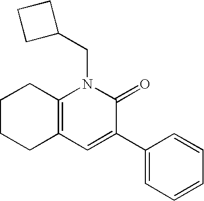

To (75 mg, 266 μmol) of intermediate A was added 1 mL of toluene and 600 μL of ethanol in a 2-dram vial. To this mixture was added phenyl boronic acid (39 mg, 319 μmol), 50 mg of tetrakis(triphenylphosphine)palladium(0) (50 mg, 43 μmol), and 1 mL of 2 M solution of sodium carbonate in water. The vial was capped and shaken at 85° C. for 2 hours. The reaction was cooled to room temperature and 2 mL H₂O and 2 mL EtOAc were added to the mixture and the vial was vortexed. The top organic layer was removed to a new vial. The aqueous layer was extracted with 1 mL of EtOAc and the organic layers were combined. The combined organic layers were extracted with 2 mL saturated NaHCO₃, dried with Na₂SO₄, filtered through celite and evaporated. The residue was purified using column chromatography on an ISCO system. A 20 minute method was used using a gradient that went from 5% EtOAc/hexane to 50% EtOAc/hexane. This yielded a light yellow oil, 1-(cyclobutylmethyl)-3-phenyl-5,6,7,8-tetrahydroquinolin-2(1H)-one. (15 mg). MS [M+H]⁺=294.18.

The compounds listed in Table 1 were prepared using the procedure outlined in the synthesis of the compound of Example 1. These compounds can be prepared using the appropriate intermediate chosen from intermediates A to C, and treating that intermediate under the above-described conditions, with the appropriate boronic acid. Example 3 is prepared by analogy starting from 1-benzyl-3-bromo-5,6,7,8-tetrahydroquinolin-2(1H)-one which is itself prepared in a similar manner to Intermediates A to C. In the case of Example 5, the boronic acid was intermediate D.

TABLE 1

| Compd # | Compound | Name | MS |
|---|---|---|---|
| 2 | 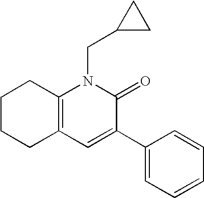 | 1-(cyclopropylmethyl)-3-phenyl-5,6,7,8-tetrahydroquinolin-2(1H)-one | [M + H]⁺ = 280.16 |
| 3 | 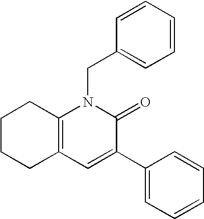 | 1-benzyl-3-phenyl-5,6,7,8-tetrahydroquinolin-2(1H)-one | [M + H]⁺ = 316.16 |
| 4 | 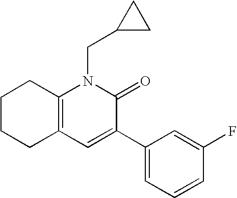 | 1-(cyclopropylmethyl)-3-(3-fluorophenyl)-5,6,7,8-tetrahydroquinolin-2(1H)-one | [M + H]⁺ = 298.15 |
| 5 | 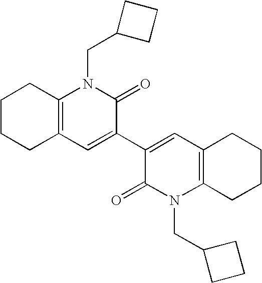 | 1,1′-bis(cyclobutylmethyl)-5,5′,6,6′,7,7′,8,8′-octahydro-3,3′-biquinoline-2,2′(1H,1′H)-dione | [M + H]⁺ = 433.28 |
| 6 | 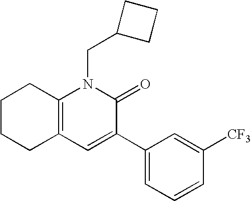 | 1-(cyclobutylmethyl)-3-(3-(trifluoromethyl)phenyl)-5,6,7,8-tetrahydroquinolin-2(1H)-one | [M + H]⁺ = 362.17 |
| 7 | 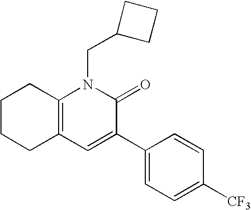 | 1-(cyclobutylmethyl)-3-(4-(trifluoromethyl)phenyl)-5,6,7,8-tetrahydroquinolin-2(1H)-one | [M + H]⁺ = 362.17 |
| 8 | 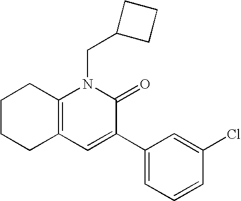 | 3-(3-chlorophenyl)-1-(cyclobutylmethyl)-5,6,7,8-tetrahydroquinolin-2(1H)-one | [M + H]⁺ = 328.14 |
| 9 | 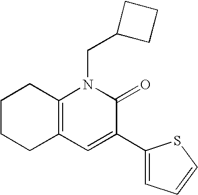 | 1-(cyclobutylmethyl)-3-(thiophen-2-yl)-5,6,7,8-tetrahydroquinolin-2(1H)-one | [M + H]⁺ = 300.13 |
| 10 | 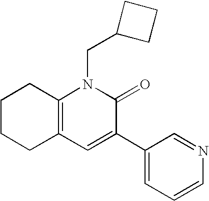 | 1-(cyclobutylmethyl)-3-(pyridin-3-yl)-5,6,7,8-tetrahydroquinolin-2(1H)-one | [M + H]⁺ = 295.17 |
| 11 | 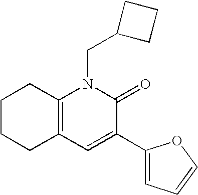 | 1-(cyclobutylmethyl)-3-(furan-3-yl)-5,6,7,8-tetrahydroquinolin-2(1H)-one | [M + H]⁺ = 284.16 |
| 12 | 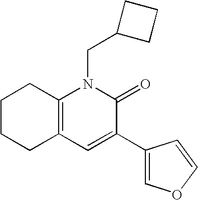 | 1-(cyclobutylmethyl)-3-(furan-3-yl)-5,6,7,8-tetrahydroquinolin-2(1H)-one | [M + H]⁺ = 284.16 |
| 13 | 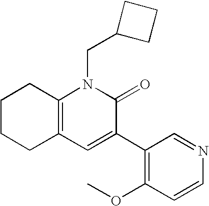 | 1-(cyclobutylmethyl)-3-(4-methoxypyridin-3-yl)-5,6,7,8-tetrahydroquinolin-2(1H)-one | [M + H]⁺ = 325.18 |
| 14 | 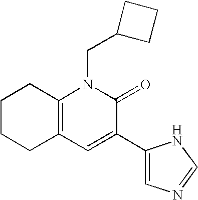 | 1-(cyclobutylmethyl)-3-(1H-imidazol-5-yl)-5,6,7,8-tetrahydroquinolin-2(1H)-one | [M + H]⁺ = 324.17 |
| 15 | 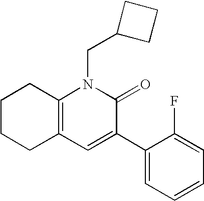 | 1-(cyclobutylmethyl)-3-(2-fluorophenyl)-5,6,7,8-tetrahydroquinolin-2(1H)-one | [M + H]⁺ = 312.17 |
| 16 | 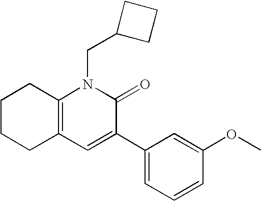 | 1-(cyclobutylmethyl)-3-(3-methoxyphenyl)-5,6,7,8-tetrahydroquinolin-2(1H)-one | [M + H]⁺ = 324.19 |
| 17 | 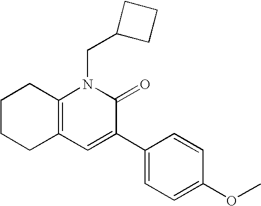 | 1-(cyclobutylmethyl)-3-(4-methoxyphenyl)-5,6,7,8-tetrahydroquinolin-2(1H)-one | [M + H]⁺ = 324.19 |
| 18 | 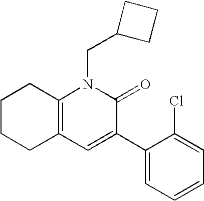 | 3-(2-chlorophenyl)-1-(cyclobutylmethyl)-5,6,7,8-tetrahydroquinolin-2(1H)-one | [M + H]⁺ = 328.14 |
| 19 | 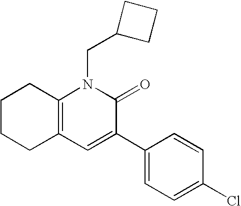 | 3-(4-chlorophenyl)-1-(cyclobutylmethyl)-5,6,7,8-tetrahydroquinolin-2(1H)-one | [M + H]⁺ = 328.14 |
| 20 | 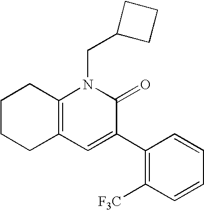 | 1-(cyclobutylmethyl)-3-(2-(trifluoromethyl)phenyl)-5,6,7,8-tetrahydroquinolin-2(1H)-one | [M + H]⁺ = 362.17 |
| 21 | 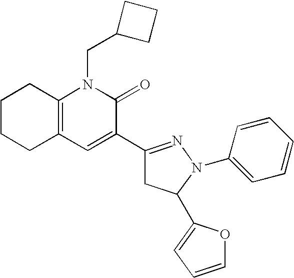 | 1-(cyclobutylmethyl)-3-(5-(furan-2-yl)-1-phenyl-4,5-dihydro-1H-pyrazol-3-yl)-5,6,7,8-tetrahydroquinolin-2(1H)-one | [M + H]⁺ = 428.23 |
| 22 | 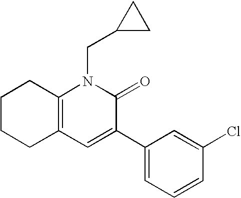 | 3-(3-chlorophenyl)-1-(cyclopropylmethyl)-5,6,7,8-tetrahydroquinolin-2(1H)-one | [M + H]⁺ = 314.12 |
| 23 | 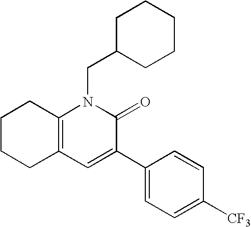 | 1-(cyclohexylmethyl)-3-(4-(trifluoromethyl)phenyl)-5,6,7,8-tetrahydroquinolin-2(1H)-one | [M + H]⁺ = 390.20 |
| 24 | 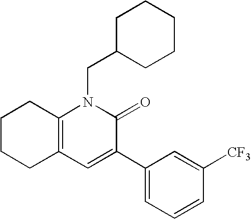 | 1-(cyclohexylmethyl)-3-(3-(trifluoromethyl)phenyl)-5,6,7,8-tetrahydroquinolin-2(1H)-one | [M + H]⁺ = 390.20 |
| 25 | 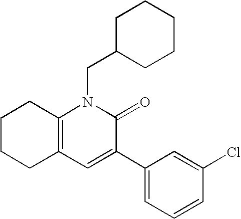 | 3-(3-chlorophenyl)-1-(cyclohexylmethyl)-5,6,7,8-tetrahydroquinolm-2(1H)-one | [M + H]⁺ = 356.17 |
| 26 | 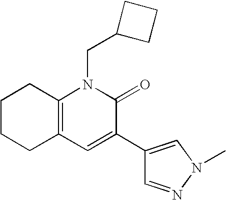 | 1-(cyclobutylmethyl)-3-(1-methyl-1H-pyrazol-4-yl)-5,6,7,8-tetrahydroquinolin-2(1H)-one | [M + H]⁺ = 298.18 |
| 27 | 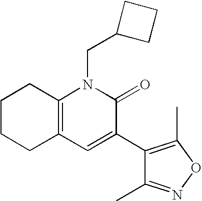 | 1-(cyclobutylmethyl)-3-(3,5-dimethylisoxazol-4-yl)-5,6,7,8-tetrahydroquinolin-2(1H)-one | [M + H]⁺ = 313.18 |
| 28 | 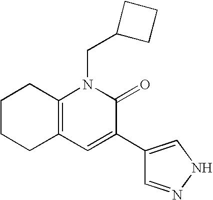 | 1-(cyclobutylmethyl)-3-(1H-pyrazol-4-yl)-5,6,7,8-tetrahydroquinolin-2(1H)-one | [M + H]⁺ = 284.17 |
| 29 | 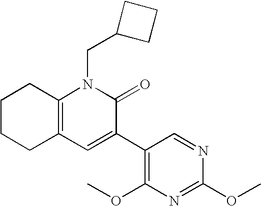 | 1-(cyclobutylmethyl)-3-(2,4-dimethoxypyrimidin-5-yl)-5,6,7,8-tetrahydroquinolin-2(1H)-one | [M + H]⁺ = 356.19 |

### Example 32

### Preparation of 1-(cyclobutylmethyl)-3-(pyridin-4-ylmethyl)-5,6,7,8-tetrahydroquinolin-2(1H)-one

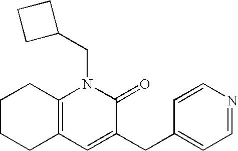

The residue was dissolved in a mixture of 300 μL of toluene and 200 μL of ethanol in a 2-dram vial. To this mixture was added 4-(bromomethyl)pyridine (24 mg, 85 μmole) as its HCl salt, tetrakis(triphenylphosphine)palladium(0) (15 mg, 13 μmole), and 300 μL of 2M solution of sodium carbonate in water. The vial was capped and shaken at 85° C. for 2 hours. The reaction was cooled to room temperature and 2 mL of H₂O and 2 mL of EtOAc were added to the mixture and the vial was vortexed. The top organic layer was removed to a new vial. The aqueous layer was extracted with 1 mL of EtOAc and the organic layers were combined. The combined organic layers were extracted with 2 mL of saturated NaHCO₃, dried with Na₂SO₄, filtered through celite and evaporated. The residue was purified using a Waters Autopurification system. A 10 minute method was used using a gradient from 70% water/acetonitrile to 10% water/acetonitrile with 0.1% formic acid as a modifier. This yielded a white solid, 1-(cyclobutylmethyl)-3-(pyridin-4-ylmethyl)-5,6,7,8-tetrahydroquinolin-2(1H)-one, (5 mg). MS [M+H]⁺=309.19.

The compounds listed in Table 2 were prepared using the procedure outlined in the synthesis of the compound of Example 30. These compounds can be prepared using the appropriate intermediate chosen from intermediates D or E, and treating that intermediate under the above-described conditions, with the appropriate bromide. In the case of example 35, the bromide was intermediate A.

TABLE 2

| Compd # | Compound | Name | MS |
|---|---|---|---|
| 30 | 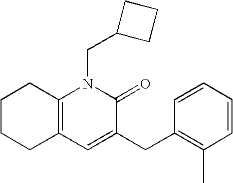 | 1-(cyclobutylmethyl)-3-(2-methylbenzyl)-5,6,7,8-tetrahydroquinolin-2(1H)-one | [M + H]⁺ = 322.21 |
| 31 | 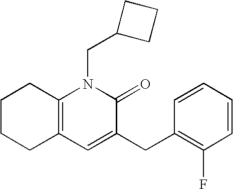 | 1-(cyclobutylmethyl)-3-(2-fluorobenzyl)-5,6,7,8-tetrahydroquinolin-2(1H)-one | [M + H]⁺ = 326.18 |
| 32 | 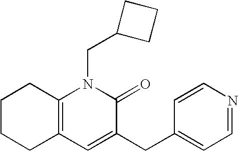 | 1-(cyclobutylmethyl)-3-(pyridin-4-ylmethyl)-5,6,7,8-tetrahydroquinolin-2(1H)-one | [M + H]⁺ = 309.19 |
| 33 | 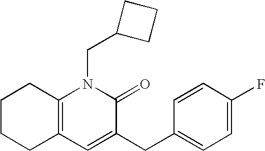 | 1-(cyclobutylmethyl)-3-(4-fluorobenzyl)-5,6,7,8-tetrahydroquinolin-2(1H)-one | [M + H]⁺ = 326.18 |
| 34 | 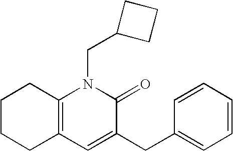 | 3-benzyl-1-(cyclobutylmethyl)-5,6,7,8-tetrahydroquinolin-2(1H)-one | [M + H]⁺ = 308.19 |

### Example 35

### Preparation of tert-butyl 3-(3-chlorophenyl)-1-(cyclobutylmethyl)-2-oxo-1,2,7,8-tetrahydro-1,6-naphthyridine-6(5H)-carboxylate

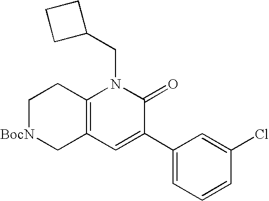

Intermediate G, tert-butyl 3-bromo-1-(cyclobutylmethyl)-2-oxo-1,2,7,8-tetrahydro-1,6-naphthyridine-6(5H)-carboxylate (25 mg, 63 μmol), and a stir bar were placed in a 0.5-2 mL microwave vial. 600 μL of THF and 300 μL of 2M Na₂CO₃ in water were added followed by 3-chlorophenyl boronic acid (11 mg, 69 μmol) and the palladium catalyst (20 mg, 17 μmol). The vial was capped and heated at 160° C. for 10 minutes in the microwave. The reaction was cooled to room temperature and 1 mL of H₂O and 1 mL of EtOAc were added to the mixture and the vial was vortexed. The top organic layer was removed to a new vial. The aqueous layer was extracted with 1 mL of EtOAc and the organic layers were combined. The combined organic layers were dried with Na₂SO₄, filtered through celite and evaporated. This was purified on the ISCO using a 4 gr column and a gradient from 10% EtOAc/Hex to 80% EtOAc/Hex. This yielded a light yellow oil, tert-butyl 3-(3-chlorophenyl)-1-(cyclobutylmethyl)-2-oxo-1,2,7,8-tetrahydro-1,6-naphthyridine-6(5H)-carboxylate, (13 mg). MS [M+H]⁺=429.19.

The compounds listed in Table 3 were prepared using the procedure outlined in the synthesis of the compound of Example 35. These compounds can be prepared using intermediate chosen from intermediates F or G, and treating that intermediate under the above-described conditions, with the appropriate boronic acid.

TABLE 3

| Compd # | Compound | Name | MS |
|---|---|---|---|
| 35 | 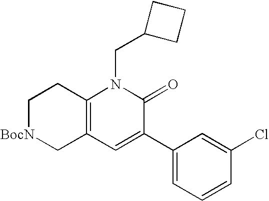 | tert-butyl 3-(3-chlorophenyl)-1-(cyclobutylmethyl)-2-oxo-1,2,7,8-tetrahydro-1,6-naphthyridine-6(5H)-carboxylate | [M + H]⁺ = 429.19 |
| 36 | 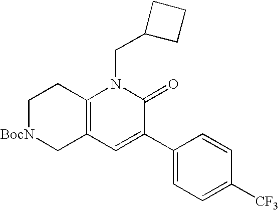 | tert-butyl 3-(4-(trifluoromethyl)phenyl)-1-(cyclobutylmethyl)-2-oxo-1,2,7,8-tetrahydro-1,6-naphthyridine-6(5H)-carboxylate | [M + H]⁺ = 463.21 |
| 37 | 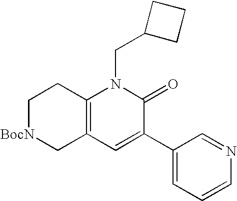 | tert-butyl 1-(cyclobutylmethyl)-2-oxo-3-(pyridin-3-yl)-1,2,7,8-tetrahydro-1,6-naphthyridine-6(5H)-carboxylate | [M + H]⁺ = 396.22 |

### Example 38

### Preparation of 3-(3-chlorophenyl)-1-(cyclobutylmethyl)-5,6,7,8-tetrahydro-1,6-naphthyridin-2(1H)-one

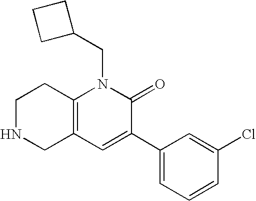

tert-butyl 3-(3-chlorophenyl)-1-(cyclobutylmethyl)-2-oxo-1,2,7,8-tetrahydro-1,6-naphthyridine-6(5H)-carboxylate (10 mg, 23 μmol) was placed in a 2 dram vial. 1 mL of 25% TFA/DCM was added. The solution fumed and turned a light yellow/green. The reaction was checked after 30 minutes by LC/MS and revealed the desired product mass and no starting material. Slow addition of saturated bicarbonate (about 4 mL) quenched the TFA and brought the reactions pH to 9. The remaining DCM was transferred to a new 2 dram vials. The aqueous layer was extracted with 2×1 mL of DCM. The combined organic layers were dried with sodium sulfate, filtered through a plug of glass wool and evaporated. This yielded a yellow oil, 3-(3-chlorophenyl)-1-(cyclobutylmethyl)-5,6,7,8-tetrahydro-1,6-naphthyridin-2(1H)-one, (6 mg). MS [M+H]⁺=329.13.

The compounds listed in Table 4 were prepared using the procedure outlined in the synthesis of the compound of Example 38. These compounds can be prepared using compounds 35-37, and treating that compound under the above-described conditions, TFA/DCM. The same conditions can be applied compounds containing differing but similar functionality at R₁ or R₂ of formula I.

TABLE 4

| Compd # | Compound | Name | MS |
|---|---|---|---|
| 38 | 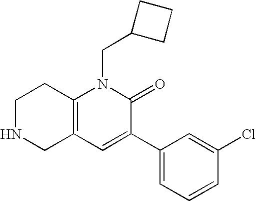 | 3-(3-chlorophenyl)-1-(cyclobutylmethyl)-5,6,7,8-tetrahydro-1,6-naphthyridin-2(1H)-one | [M + H]⁺ = 329.13 |
| 39 | 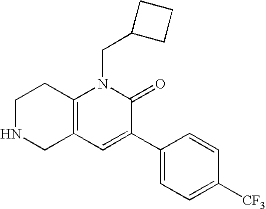 | 3-(4-(trifluoromethyl)phenyl)-1-(cyclobutylmethyl)-5,6,7,8-tetrahydro-1,6-naphthyridin-2(1H)-one | [M + H]⁺ = 363.16 |
| 40 | 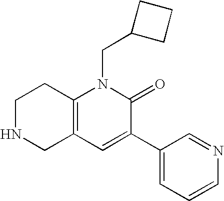 | 1-(cyclobutylmethyl)-3-(pyridin-3-yl)-5,6,7,8-tetrahydro-1,6-naphthyridin-2(1H)-one | [M + H]⁺ = 296.17 |

### Example 41

### Preparation of 3-(3-chlorophenyl)-1-(cyclobutylmethyl)-6-(methylsulfonyl)-5,6,7,8-tetrahydro-1,6-naphthyridin-2(1H)-one

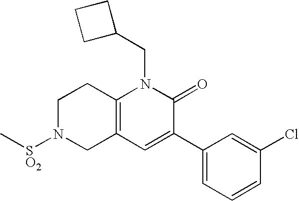

3-(3-chlorophenyl)-1-(cyclobutylmethyl)-5,6,7,8-tetrahydro-1,6-naphthyridin-2(1H)-one (3.2 mg, 10 μmol) was taken up in 0.5 mL of DCM. To the vial, DIPEA (5 uL, 30 umol) was added followed by methanesulfonyl chloride (2.3 μL, 30 μmol). The vial was capped and shaken at room temperature for 1 hour. LCMS showed one main peak having the desired mass. The reaction mixture was extracted with 1 mL of saturated bicarbonate. The organic layer was transferred to a new vial and the water layer was extracted with 1 mL DCM. The organic layers were combined, dried with sodium sulfate, filtered through a plug of glass wool and evaporated. This yielded 3-(3-chlorophenyl)-1-(cyclobutylmethyl)-6-(methylsulfonyl)-5,6,7,8-tetrahydro-1,6-naphthyridin-2(1H)-one (1.0 mg). MS [M+H]⁺=407.11.

The compounds listed in Table 5 were prepared using the procedure outlined in the synthesis of the compound of Example 41. These compounds can be prepared using the appropriate such as examples 38-40 or compounds containing differing but similar functionality at R₁ or R₂ of formula I, and treating that intermediate under the above-described conditions, with the appropriate acid chloride or sulfonyl chloride.

TABLE 5

| Compd # | Compound | Name | MS |
|---|---|---|---|
| 41 | 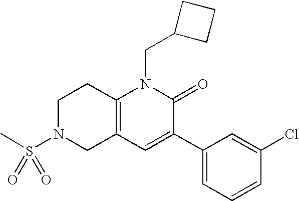 | 3-(3-chlorophenyl)-1-(cyclobutylmethyl)-6-(methylsulfonyl)-5,6,7,8-tetrahydro-1,6-naphthyridin-2(1H)-one | [M + H]⁺ = 407.11 |
| 42 | 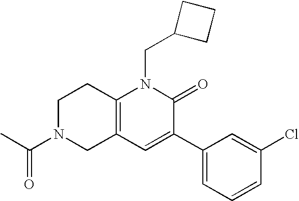 | 6-acetyl-3-(3-chlorophenyl)-1-(cyclobutylmethyl)-5,6,7,8-tetrahydro-1,6-naphthyridin-2(1H)-one | [M + H]⁺ = 371.14 |
| 43 | 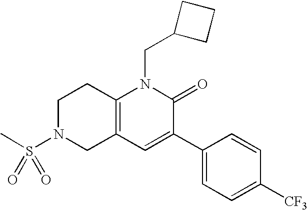 | 1-(cyclobutylmethyl)-6-(methylsulfonyl)-3-(4-(trifluoromethyl)phenyl)-5,6,7,8-tetrahydro-1,6-naphthyridin-2(1H)-one | [M + H]⁺ = 441.14 |
| 44 | 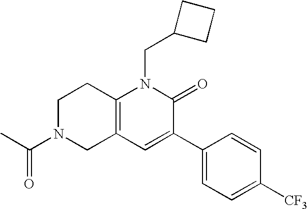 | 6-acetyl-1-(cyclobutylmethyl)-3-(4-(trifluoromethyl)phenyl)-5,6,7,8-tetrahydro-1,6-naphthyridin-2(1H)-one | [M + H]⁺ = 405.17 |
| 45 | 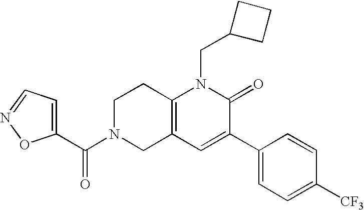 | 1-(cyclobutylmethyl)-6-(isoxazole-5-carbonyl)-3-(4-(trifluoromethyl)phenyl)-5,6,7,8-tetrahydro-1,6-naphthyridin-2(1H)-one | [M + H]⁺ = 458.16 |
| 46 | 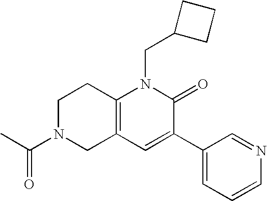 | 6-acetyl-1-(cyclobutylmethyl)-3-(pyridin-3-yl)-5,6,7,8-tetrahydro-1,6-naphthyridin-2(1H)-one | [M + H]⁺ = 338.18 |
| 47 | 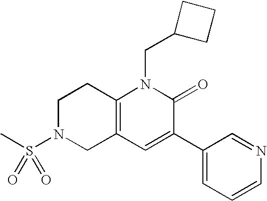 | 1-(cyclobutylmethyl)-6-(methylsulfonyl)-3-(pyridin-3-yl)-5,6,7,8-tetrahydro-1,6-naphthyridin-2(1H)-one | [M + H]⁺ = 374.15 |
| 48 | 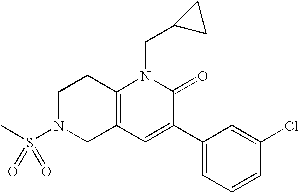 | 3-(3-chlorophenyl)-1-(cyclopropylmethyl)-6-(methylsulfonyl)-5,6,7,8-tetrahydro-1,6-naphthyridin-2(1H)-one | [M + H]⁺ = 393.10 |
| 49 | 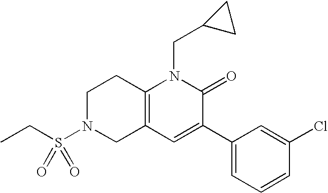 | 3-(3-chlorophenyl)-1-(cyclopropylmethyl)-6-(ethylsulfonyl)-5,6,7,8-tetrahydro-1,6-naphthyridin-2(1H)-one | [M + H]⁺ = 407.11 |
| 50 | 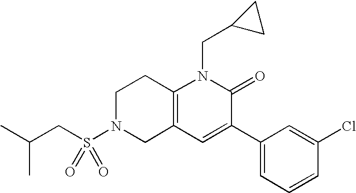 | 3-(3-chlorophenyl)-1-(cyclopropylmethyl)-6-(isobutylsulfonyl)-5,6,7,8-tetrahydro-1,6-naphthyridin-2(1H)-one | [M + H]⁺ = 435.14 |
| 51 | 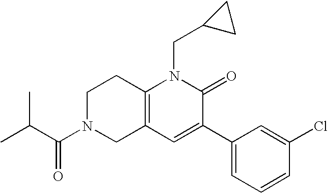 | 3-(3-chlorophenyl)-1-(cyclopropylmethyl)-6-isobutyryl-5,6,7,8-tetrahydro-1,6-naphthyridin-2(1H)-one | [M + H]⁺ = 385.16 |
| 52 | 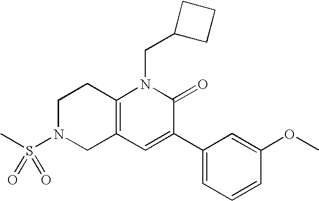 | 1-(cyclobutylmethyl)-3-(3-methoxyphenyl)-6-(methylsulfonyl)-5,6,7,8-tetrahydro-1,6-naphthyridin-2(1H)-one | [M + H]⁺ = 403.16 |
| 53 | 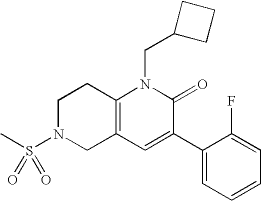 | 1-(cyclobutylmethyl)-3-(2-fluorophenyl)-6-(methylsulfonyl)-5,6,7,8-tetrahydro-1,6-naphthyridin-2(1H)-one | [M + H]⁺ = 391.14 |
| 54 | 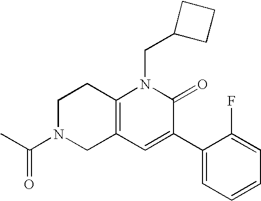 | 6-acetyl-1-(cyclobutylmethyl)-3-(2-fluorophenyl)-5,6,7,8-tetrahydro-1,6-naphthyridin-2(1H)-one | [M + H]⁺ = 355.17 |
| 55 | 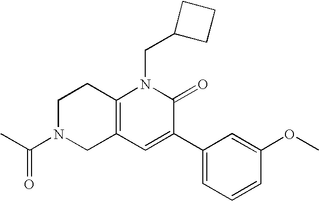 | 6-acetyl-1-(cyclobutylmethyl)-3-(3-methoxyphenyl)-5,6,7,8-tetrahydro-1,6-naphthyridin-2(1H)-one | [M + H]⁺ = 367.19 |
| 56 | 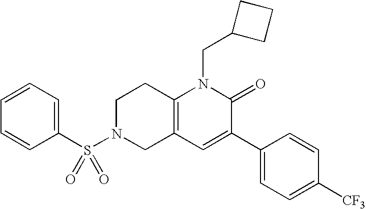 | 1-(cyclobutylmethyl)-6-(phenylsulfonyl)-3-(4-(trifluoromethyl)phenyl)-5,6,7,8-tetrahydro-1,6-naphthyridin-2(1H)-one | [M + H]⁺ = 503.16 |
| 57 | 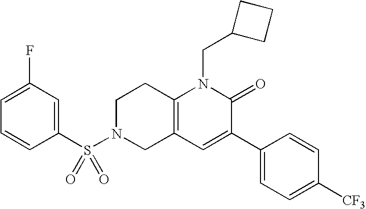 | 1-(cyclobutylmethyl)-6-(3-fluorophenylsulfonyl)-3-(4-(trifluoromethyl)phenyl)-5,6,7,8-tetrahydro-1,6-naphthyridin-2(1H)-one | [M + H]⁺ = 521.14 |

### Example 58

### Preparation of 1-(cyclobutylmethyl)-3-(2-fluorobenzyl)-6-(methylsulfonyl)-5,6,7,8-tetrahydro-1,6-naphthyridin-2(1H)-one

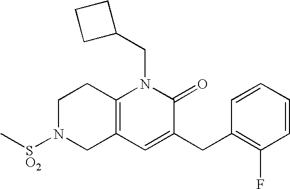

### Step 1: Preparation of 6-(tert-butoxycarbonyl)-1-(cyclobutylmethyl)-2-oxo-1,2,5,6,7,8-hexahydro-1,6-naphthyridin-3-ylboronic acid

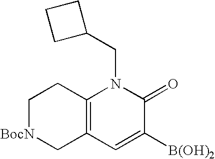

tert-butyl 3-bromo-1-(cyclobutylmethyl)-2-oxo-1,2,7,8-tetrahydro-1,6-naphthyridine-6(5H)-carboxylate (20 mg, 50 μmol) was dissolved in 1 ml of THF in a 2-dram vial containing a stir bar. The vial was cooled to −75° C. using a dry ice/acetone bath. To this mixture was added 2.5M n-BuLi (22 μL, 55 μmol). After the reaction was stirred for 1 hr. at −75° C., tributyl borate (64 μL, 55 μmol) was added and the reaction was allowed to warm to room temperature. The reaction was hydrolyzed with 1 mL of 10% HCl/water. The reaction mixture was extracted twice with 1 mL of EtOAc. The combined organic layers were dried with Na₂SO₄, filtered and evaporated. The resulting brown oil was used without purification. MS [M+H]⁺=363.20.

### Step 2: Preparation of tert-butyl 1-(cyclobutylmethyl)-3-(2-fluorobenzyl)-2-oxo-1,2,7,8-tetrahydro-1,6-naphthyridine-6(5H)-carboxylate

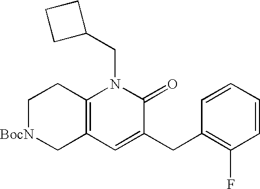

The residue from Step 1 was dissolved in a mixture of 300 μL of toluene and 200 μL of ethanol in a 2-dram vial. To this mixture was added (24 mg, 85 μmole) of 2-fluorobenzyl bromide, tetrakis(triphenylphosphine)palladium(0) (15 mg, 13 umol), and 300 μL of 2M solution of sodium carbonate in water. The vial was capped and shaken at 85° C. for 2 hours. The reaction was cooled to room temperature and 2 mL of H₂O and 2 mL of EtOAc were added to the mixture and the vial was vortexed. The top organic layer was removed to a new vial. The aqueous layer was extracted with 1 mL of EtOAc and the organic layers were combined. The combined organic layers were extracted with 2 mL of saturated NaHCO₃, dried with Na₂SO₄, filtered through celite and evaporated. This was purified on the ISCO using a 4 gr column and a gradient from 10% EtOAc/Hex to 80% EtOAc/Hex. The appropriate fractions were combined and the volatiles evaporated. This yielded a light yellow oil tert-butyl 1-(cyclobutylmethyl)-3-(2-fluorobenzyl)-2-oxo-1,2,7,8-tetrahydro-1,6-naphthyridine-6(5H)-carboxylate (5 mg). MS [M+H]⁺=427.23.

### Step 3: Preparation of 1-(cyclobutylmethyl)-3-(2-fluorobenzyl)-5,6,7,8-tetrahydro-1,6-naphthyridin-2(1H)-one

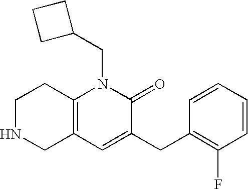

tert-butyl 1-(cyclobutylmethyl)-3-(2-fluorobenzyl)-2-oxo-1,2,7,8-tetrahydro-1,6-naphthyridine-6(5H)-carboxylate (10 mg, 23 μmol) was placed in a 2 dram vial. 1 mL of 25% TFA/DCM was added. The solution fumed and turned a light yellow/green. The reaction was checked after 30 minutes by LC/MS and revealed the desired product mass and no starting material. Slow addition of saturated bicarbonate (about 4 mL) quenched the TFA and brought the reactions pH to 9. The remaining DCM was transferred to a new 2 dram vials. The aqueous layer was extracted with 2×1 mL of DCM. The combined organic layers were dried with sodium sulfate, filtered through a plug of glass wool and evaporated. This yielded a yellow oil, 1-(cyclobutylmethyl)-3-(2-fluorobenzyl)-5,6,7,8-tetrahydro-1,6-naphthyridin-2(1H)-one, (6 mg) that was used without further purification. MS [M+H]⁺=327.18.

### Step 4: Preparation of 1-(cyclobutylmethyl)-3-(2-fluorobenzyl)-6-(methylsulfonyl)-5,6,7,8-tetrahydro-1,6-naphthyridin-2(1H)-one

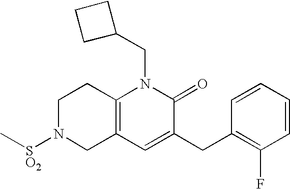

1-(cyclobutylmethyl)-3-(2-fluorobenzyl)-5,6,7,8-tetrahydro-1,6-naphthyridin-2(1H)-one (2 mg, 6 μmol) was taken up in 0.5 mL of solution transferred to a 2 dram vial. To the vial, DIPEA (3 μl, 18 μmol) was added followed by methanesulfonyl chloride (5 μl, 60 μmol). The vial was capped and shaken at room temperature for 1 hour. LCMS showed one main peak having the desired mass. The reaction mixture was extracted with 1 mL of saturated bicarbonate. The organic layer was transferred to a new vial and the water layer was extracted with 1 mL DCM. The organic layers were combined, dried with sodium sulfate, filtered through a plug of glass wool and evaporated. This yielded a yellow oil, 1-(cyclobutylmethyl)-3-(2-fluorobenzyl)-6-(methylsulfonyl)-5,6,7,8-tetrahydro-1,6-naphthyridin-2(1H)-one (2.0 mg). MS [M+H]⁺=405.16.

### Screening Methods

The ability of compounds to act as agonists or inverse agonists at human CB2 and CB1 receptors (hCB2, hCB1, respectively) was determined by measuring changes in intracellular cAMP levels. Chinese Hamster Ovary (CHO-K1) cell lines stably expressing hCB2 (Genebank: X74328) or hCB1 (Genebank: X54937) were purchased from Euroscreen (Gosselies, Belgium).

Cell lines were grown in suspension in EX-CELL 302 CHO Serum-free medium (Sigma, cat #14324C) supplemented with 1% Fetal Bovine Serum, glutamine and non-essential amino-acids under 0.4 mg/mL G418 selection.

Receptor mediated responses were determined by measuring changes in intracellular cAMP using LANCE cAMP detection kit (cat #AD0264, PerkinElmer, Wellesley, Mass.) based on time-resolved fluorescence resonance energy transfer (TR-FRET). Changes in cAMP were determined in cells pre-incubated with IBMX (isobutyl methylxanthine) and prestimulated with NKH-477 (a water soluble forskolin derivative, cat #1603, Tocris, Ellisville, Mo.) to increase basal cAMP levels as detailed below.

On the day of the experiment, cells were spun at low speed for 5 min at room temperature. The supernatant was removed and cells were resuspended in stimulation buffer (Hanks Buffered Salt Solution/5 mM HEPES, containing 0.5 mM IBMX (cat #17018, Sigma) and 0.02% BSA (PerkinElmer, cat #CR₈₄-100)). Cell clumps were removed by filtering through cell strainer 40 μm (BD Falcon, Discovery Labware, Bedford, Mass.) and diluted to 2×10⁵ cells/mL. Antibody supplied with the LANCE cAMP immunoassay kit was then added according to the manufacturer's instructions. An aliquot of cells was taken for un-induced controls. To the remaining cells was added NKH-477 (a water soluble forskolin derivative, Tocris cat #1603) to a final concentration of 2-8 μM. Cells were then incubated for 30 min at room temperature prior to adding to Proxiplates containing test compounds (final DMSO concentration was less than 0.5%) with a Multidrop bulk dispenser, followed by a sixty minute incubation at room temperature. The response was stopped by addition of the detection mix supplied with the LANCE kit.

The reagents were allowed to equilibrate for three hours prior to reading on an Envision multi-mode detector (PerkinElmer). TR-FRET was measured using a 330-380 nm excitation filter, a 665 nm emission filter, dichroic mirror 380 nm and Z=1 mm.

Cyclic AMP concentrations in each well were back-calculated from a cAMP standard curve run concurrently during each assay. Each plate contained 16 wells of forskolin stimulated cells and 16 wells of forskolin plus CP55,940-treated. CP55,940-treated cells were treated with CP55,940 (Tocris cat. # 0949) at 1 μm and the maximal response was used as the full range (100%) standard. WIN55,212 (Tocris cat. # 1038) was used as an internal standard. Concentrations of cAMP were expressed as a percent of the difference of these two groups of wells. Concentration-response data including ECso (the concentration of compound producing 50% of the maximal response) and intrinsic activity (the percent maximal activation compared to full activation by CP55,940) were determined using a four-parameter non-linear regression algorithm (Xlfit equation 251, IDBS). Results for compounds I-58 are shown in Table 7 below:

TABLE 7

| Compound | hCB2 | rCB2 | hCB1 |
|---|---|---|---|
| 1 | +B | +A | AR |
| 2 | +B | +C | AR |
| 3 | AR | AR | AR |
| 4 | 4 | +C | +C |
| 5 | +A | +A | +C |
| 6 | +A | +A | AR |
| 7 | +A | +A | AR |
| 8 | +A | +A | AR |
| 9 | +B | +C | AR |
| 10 | +B | +B | AR |
| 11 | +B | +B | AR |
| 12 | +C | +B | AR |
| 13 | +B | AR | AR |
| 14 | AR | AR | AR |
| 15 | +B | +B | AR |
| 16 | +B | +B | AR |
| 17 | AR | AR | AR |
| 18 | +B | +B | AR |
| 19 | +B | +B | AR |
| 20 | +B | +B | AR |
| 21 | +C | AR | AR |
| 22 | +B | −B | AR |
| 23 | +B | −B | +B |
| 24 | +A | AR | +B |
| 25 | +B | +A | +B |
| 26 | +B | +A | AR |
| 27 | +B | AR | AR |
| 28 | AR | −C | AR |
| 29 | +B | +A | AR |
| 30 | +A | +A | AR |
| 31 | +A | +A | AR |
| 32 | +B | +B | AR |
| 33 | +B | +B | AR |
| 34 | +B | +B | AR |
| 35 | AR | −B | AR |
| 36 | AR | −C | AR |
| 37 | AR | −C | AR |
| 38 | AR | AR | AR |
| 39 | +C | +C | AR |
| 40 | +C | +B | AR |
| 41 | +A | +A | AR |
| 42 | +A | +A | +C |
| 43 | +A | +B | AR |
| 44 | +B | +B | AR |
| 45 | AR | −C | AR |
| 46 | +B | +B | AR |
| 47 | AR | AR | AR |
| 48 | +A | +A | AR |
| 49 | +B | +B | AR |
| 50 | +C | −B | −B |
| 51 | +B | AR | AR |
| 52 | +B | +A | AR |
| 53 | +A | +A | AR |
| 54 | +B | +A | AR |
| 55 | AR | −C | −B |
| 56 | AR | −C | −B |
| 57 | AR | −B | AR |
| 58 | +A | +A | +B |

AR: Above assay range;
A: EC₅₀ below 100.0 nM; B: EC₅₀ in the range 100.1 nM-1 μM; C: EC₅₀ in the range 1.01 μM-10 μM;
“+” or “−”: identifies the compound as an agonist or an inverse agonist, respectively.

The examples provided herein are for illustration purposes only and are not intended to be interpreted as limiting the scope of the invention, the full scope of which will be immediately recognized by those of skill in the art.

We claim:

1. A compound having the structure according to formula I, 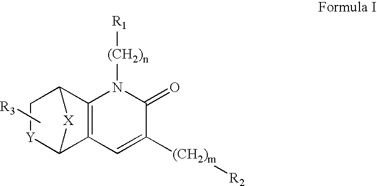
or a stereoisomer, mixture of stereoisomers, prodrug, pharmaceutically acceptable salt, hydrate, solvate, acid salt hydrate, or isomorphic crystalline form thereof,
wherein
R₁ is —H, optionally substituted C₁-C₆ alkyl, optionally substituted C₃-C₆ cycloalkyl, optionally substituted C₃-C₆ cycloalkyloxy, SO₂R₄, COR₄, optionally substituted phenyl, optionally substituted naphthyl; wherein R₄ is optionally substituted C₁-C₄ alkyl, a saturated or unsaturated optionally substituted 5-, 6- or 7-membered heterocycle optionally fused with a C₄-C₈ carbocycle, optionally substituted alkoxy;
R₂ is optionally substituted phenyl, optionally substituted C₃-C₇ cycloalkyl, optionally substituted C₃-C₇ cycloalkenyl, optionally substituted five- or six-membered heterocyclyl optionally fused with a C₄-C₈ carbocycle, optionally substituted alkoxy, or R₂ is 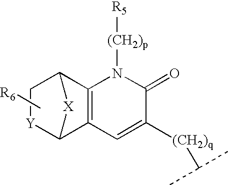
wherein R₅ is independently selected from R₁, R₆ is independently selected from R₃; and n, m, p and q are each independently 0, 1, 2, or 3;
R₃ is —H, —NH₂, optionally substituted C₁-C₆ alkyl, optionally substituted C₁-C₆ alkoxy, C₁-C₆ alkylthio, C₃-C₆ cycloalkyl, optionally substituted phenyl, optionally substituted naphthyl, optionally substituted heterocyclyl, SO₂R₇, CONHR₇, COR₇ or COOR₇, wherein R₇ is C₁-C₄ alkyl, phenyl, naphthyl or 5- or 6-membered heterocyclyl;
wherein each substituted moiety is substituted with one, two or three substituents, wherein each substituent is independently selected from the group consisting of C₁-C₆ alkyl, C₁-C₆ alkoxy, alkylthio, phenyl, heterocyclyl, halo, —OH, —NH₂, oxo, —NO₂, —CN, —COOH, and amidino, except that oxo is not permitted as a substituent of phenyl or naphthyl;
—X— is —CH₂—, —O—, —S—, —SO—, —SO₂—, or is absent; and
Y is C or N.

2. The compound according to claim 1, wherein R₁ is optionally substituted C₃-C₆ cycloalkyl.

3. The compound according to claim 2, wherein R₁ is selected from the group consisting of cyclopropyl, cyclobutyl, cyclopentyl or cyclohexyl.

4. The compound according to claim 1, wherein R₁ is C₁-C₆ alkyl.

5. The compound according to claim 1, wherein R₁ is optionally substituted phenyl.

6. The compound according to claim 1, wherein R₂ is —H, optionally substituted phenyl, optionally substituted naphthyl or optionally substituted five- or six-membered heterocyclyl.

7. The compound according to claim 6, wherein R₂ is selected from the group consisting of phenyl, thiophenyl, halophenyl, methylphenyl, methoxyphenyl, halomethylphenyl, furanyl, pyridyl, pyrazyl and pyrimidyl.

8. The compound according to claim 1, wherein R₃ is selected from the group consisting of —H, C₁-C₆ alkyl, C₁-C₆ alkoxy, C₃-C₆ cycloalkyl, phenyl, 5- or 6-membered heterocyclyl, SO₂R₇, COR₇ or COOR₇, wherein R₇ is C₁-C₄ alkyl, C₃-C₆ cycloalkyl, optionally substituted phenyl, and 5- or 6-membered heterocyclyl.

9. The compound according to claim 8, wherein R₃ is selected from the group consisting of —H, phenyl, acetyl, methylsulfonyl, ethylsulfonyl, butylsulfonyl, phenylsulfonyl, fluorophenylsulfonyl, butyloxycarbonyl, oxazolylcarbonyl and butyryl.

10. The compound according to claim 1, wherein —X— is absent.

11. The compound according to claim 1, wherein Y is N.

12. The compound according to any of claims 1-11, wherein R₃ is bonded to Y.

13. The compound according to claim 1, wherein n is zero.

14. The compound according to claim 1, wherein n is 1.

15. The compound according to claim 1, wherein m is zero.

16. The compound according to claim 1, wherein m is 1.

17. The compound according to claim 1, which binds a mammalian cannabinoid-2 receptor.

18. The compound according to claim 17, wherein the mammalian cannabinoid-2 receptor is a human cannabinoid-2 receptor.

19. The compound according to claim 18, wherein the compound is an agonist for a human cannabinoid-2 receptor.

20. The compound according to claim 19, wherein the compound has an EC₅₀ of less than about 1 uM for a human cannabinoid-2 receptor.

21. The compound according to claim 20, wherein the compound has an EC₅₀ of less than about 100 nM for a human cannabinoid-2 receptor.

22. The compound according to claim 21, wherein the compound has an EC₅₀ of less than about 10 nM for a human cannabinoid-2 receptor.

23. The compound according to claim 19, wherein the compound has an EC₅₀ of greater than about 10 mM for a human cannabinoid-1 receptor.

24. The compound according to claim 1, selected from the group consisting of

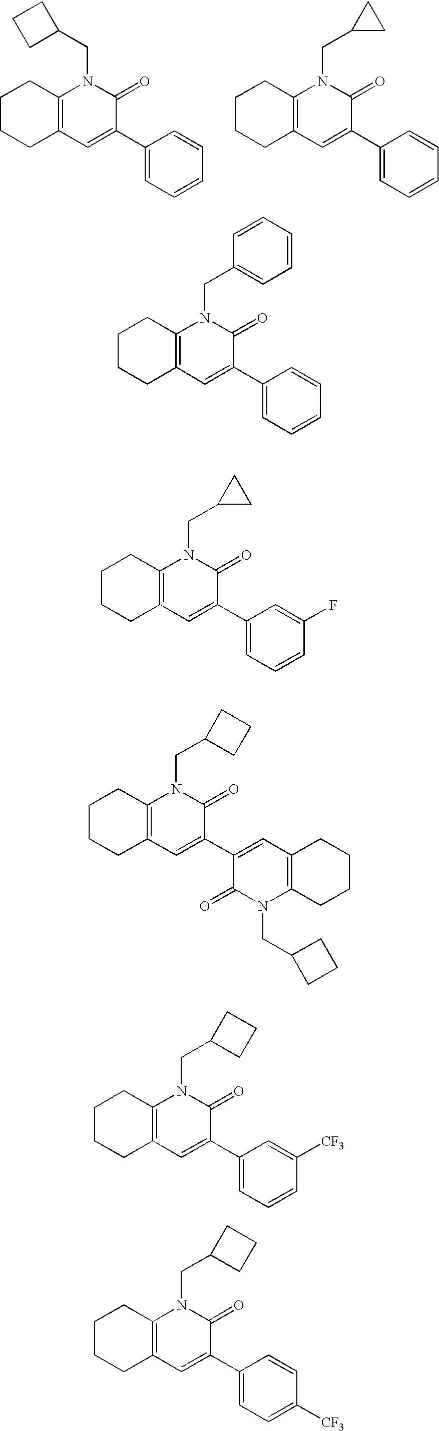

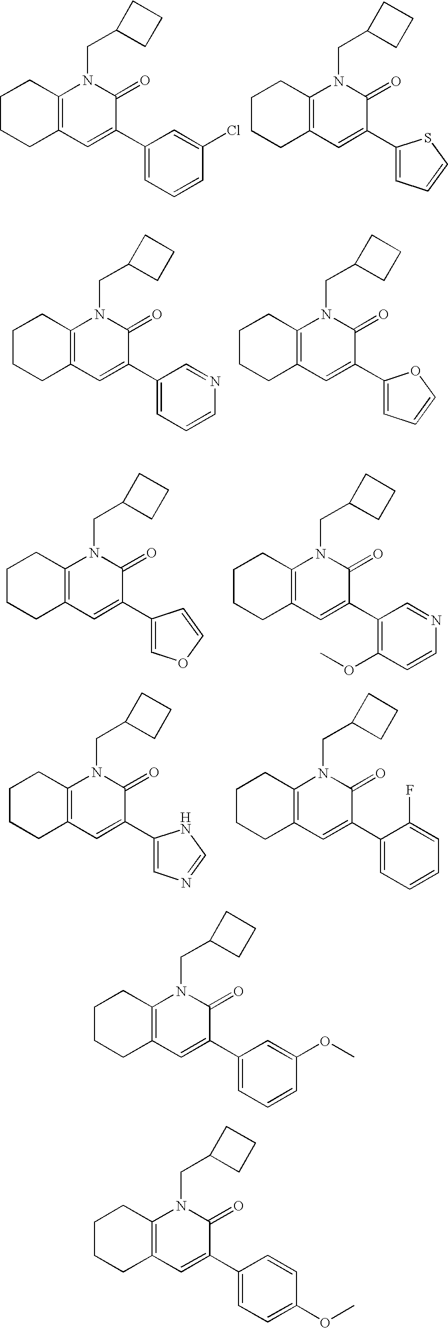

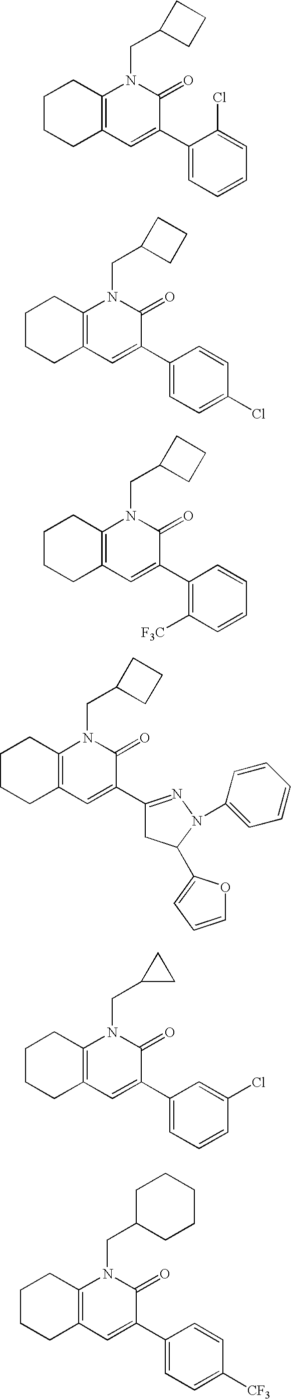

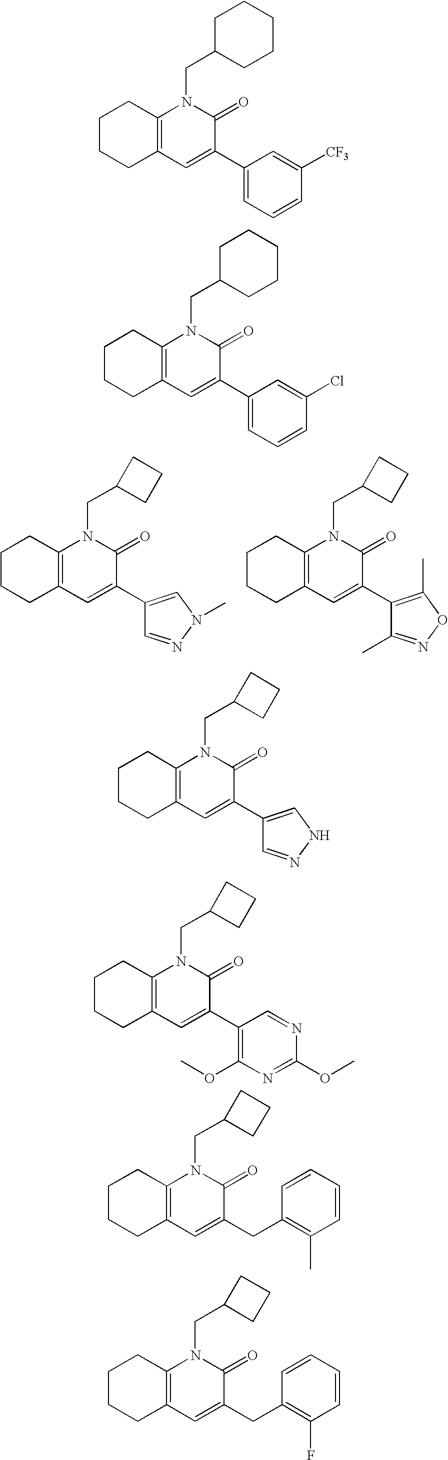

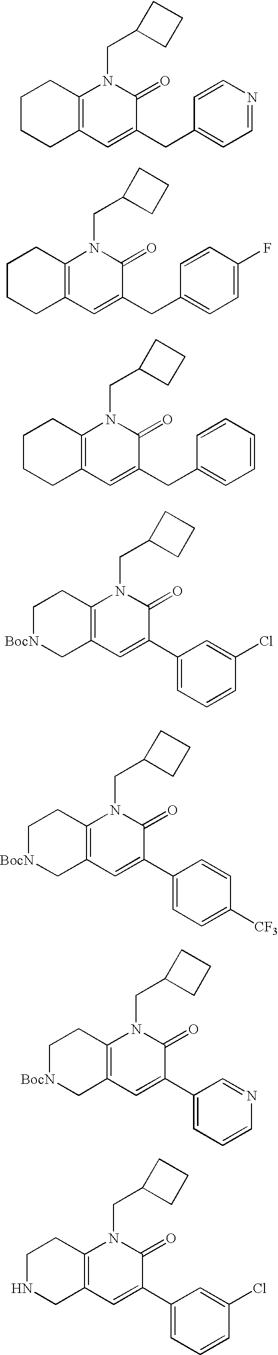

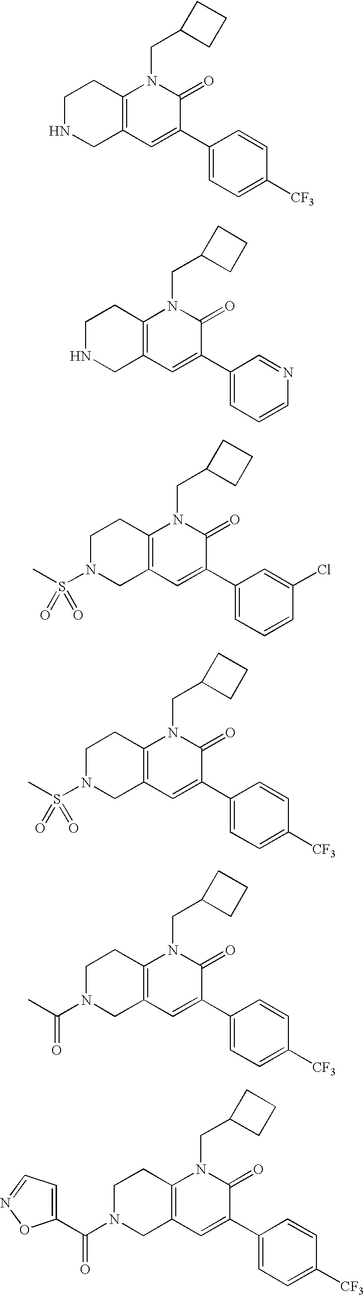

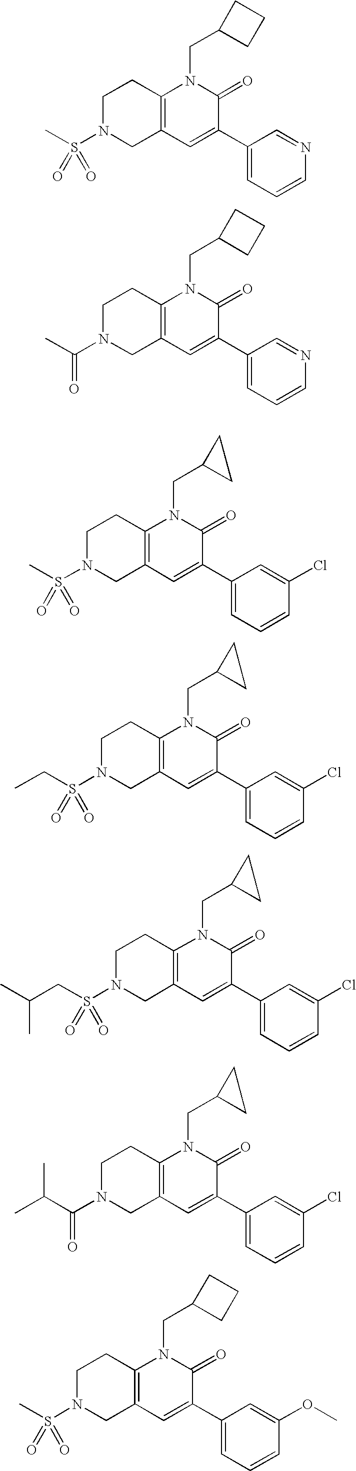

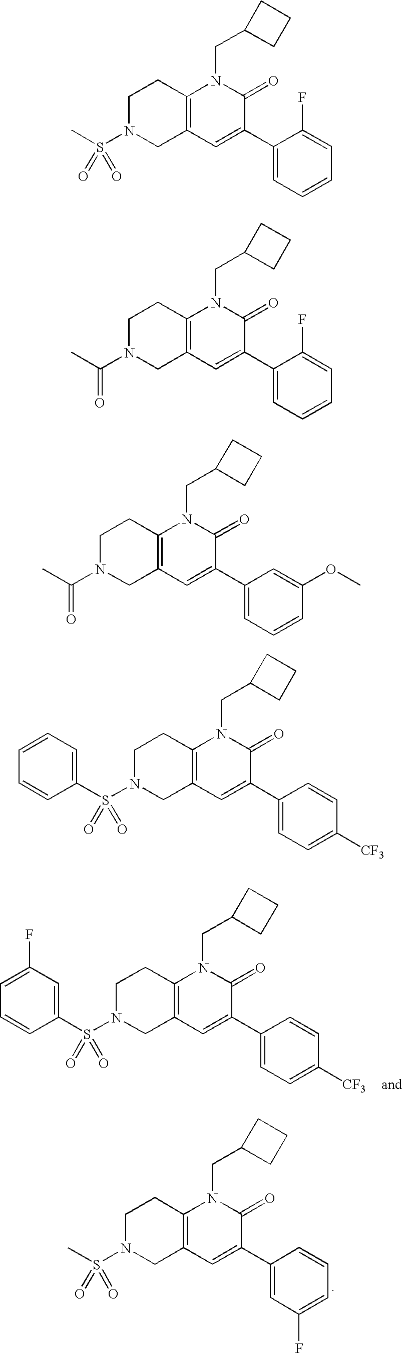

25. A pharmaceutical composition comprising a compound according to claim 1 and a pharmaceutically acceptable vehicle, carrier or diluent.
